(19) United States
(12) Patent Application Publication
(10) Pub. No.: US 2013/0137140 A1
(43) Pub. Date: May 30, 2013

(54) INCREASED PROTEIN EXPRESSION THROUGH INCREASED MEMBRANE FORMATION

(71) Applicants: Nico L.M. Callewaert, Nevele (BE); Mouna Guerfal, Veltem-Beisem (BE)

(72) Inventors: Nico L.M. Callewaert, Nevele (BE); Mouna Guerfal, Veltem-Beisem (BE)

(73) Assignees: UNIVERSITEIT GENT, Gent (BE); VIB VZW, Gent (BE)

(21) Appl. No.: 13/704,973

(22) PCT Filed: Jun. 15, 2011
(86) PCT No.: PCT/EP2011/059959

§ 371 Date: Feb. 5, 2013

(30) Foreign Application Priority Data
Jun. 17, 2010 (EP) 10166271.6

(51) Int. Cl. C12N 15/81; C12P 21/00

(52) U.S. Cl. CPC C12N 15/81; C12P 21/00
USPC 435/69.1; 435/254.21; 435/254.23; 435/254.2

(57) ABSTRACT

The present application relates to the field of protein expression technologies. More specifically, it is demonstrated that cells with increased levels of phosphatidic acid, which can be achieved through either or both of phosphatidic acid inhibition and diacylglycerol kinase overexpression, display increased membrane formation from which increased levels of proteins can be recovered.

### FIELD OF THE INVENTION

The present application relates to the field of protein expression technologies. More specifically, it is demonstrated that cells with increased levels of phosphatidic acid, which can be achieved through either or both of phosphatidic acid inhibition and diacylglycerol kinase overexpression, display increased membrane formation from which increased levels of proteins can be recovered.

### BACKGROUND

The last years an increasing interest has grown to isolate and characterize membrane proteins. Consortia have been created which study is entirely focused on the expression of membrane proteins and this in different expression systems. One example is the MEPNET (Membrane Protein Network) consortium. The high interest in membrane proteins is due to the fact that they play an important role in several biological processes such as ion transport, recognition of molecules, signal transduction etc. GPCRs (G-protein coupled receptors) are the major class of pharmacological interesting membrane proteins. These proteins with 7 transmembrane regions are involved in recognition of signals as diverse as light (rhodopsin), smells (olfactory receptors), neurotransmitters and hormones. The human genome, for instance, encodes hundreds of these receptors and in view of their crucial role in the ‘sensing’ of numerous signals and because of their accessible localization on the surface of the membrane, these proteins are the target of a variety of pharmacological compounds. More than 40% of the drugs on the market are directed to specific GPCRs.

Although membrane proteins represent 20-30% of all genes in prokaryotes as well as in eukaryotes, only little is known about structure and function relationship of membrane proteins. One reason is their low abundance in native tissue leading to the fact that not enough material can be isolated for structural studies. By way of example, only for six GPCRs the structure has been characterized, two of which in the last year: rhodopsin, the β1 and β2 adrenergic receptors, the adenosine 2A receptor, the CXCR4 receptor and the dopamine D3 receptor. It is no coincidence that exactly these 6 belong to the small group of easily obtainable GPCRs. Rhodopsin was purified from cow eyes, where it naturally occurs in high concentrations—a lone exception to the rule of low expression levels—and as a result it was the first GPCR for which a structure was determined (Palczewski et al., 2000; Salom et al., 2006). The β1 and β2 adrenergic receptors and the adenosine 2A receptors were known to those of skill in the art to be intrinsically fairly easily expressible (Lundstrom et al., 2006) and their structures were published in 2007-2008 (Cherezov et al., 2007; Rasmussen et al., 2007; Jaakola et al., 2008; Warne et al., 2008). The two most recent crystal structures are those of the CXCR4 receptor (Wu et al., 2010) and the dopamine D3 receptor (Chien et al., 2010). Most GPCRs however, express at levels that are 10 to 1000-fold lower than those of the crystallized receptors in non-engineered eukaryotic cells. Consequently, fundamental biotechnological work is necessary to increase the expression levels for the majority of these receptors to those required for biophysical characterization.

Indeed, techniques used to obtain high resolution 3D structures such as NMR and X-ray crystallography, require large amounts (milligrams) of purified proteins. A challenge therefore is to produce these proteins to levels high enough to allow structural studies. GPCRs have been successfully overexpressed in bacteria, yeast, mammalian cell lines and insect cells. Expression levels although are still rather low and for most proteins a 5 to 10-fold increase in expression level would lead to sufficient material to allow isolation of the protein for structural studies.

Expression of eukaryotic membrane proteins in prokaryotic systems mostly leads to poor expression levels and in many cases the protein ends up in denatured form in inclusion bodies. Expression of the rat neurotensin receptor (Grisshammer et al., 1993) and the human adenosine A2A receptor are examples of the few successfully overexpressed GPCRs in E. coli. Expression in yeast is a valuable alternative for the expression of membrane proteins. Yeast cells are easy to handle, and can grow in fermentors to very high cell densities. Different techniques to increase the expression levels of the membrane protein have been used in yeast such as lowering the induction temperature, adding antagonist DMSO or histidine to the induction medium (André et al., 2006). None of these techniques however markedly increased the total yield of the expressed protein but improved significantly the functionality of the protein. This is reflected by the fact that only very few of these membrane proteins have been crystallized, even though 30% of eukaryotic genes code for these proteins. Only three yeast species are currently used for the production of membrane proteins, namely Saccharomyces cerevisiae, Schizosaccharomyces pombe and Pichia pastoris. P. pastoris is the most successfully used yeast which is reflected in the amount of membrane protein structures present in the membrane protein data base.

Another yeast of biotechnological importance is Yarrowia lipolytica. It has good capacities for the secretion of heterologous proteins as shown by the number of proteins expressed in this yeast (Madzak et al., 2004). Yarrowia lipolytica is an oleaginous yeast isolated predominantly from lipid- or protein containing sources such as cheese, yoghurts, kefir and meat. It is a dimorphic fungus capable to grow as a yeast cell or hyphae or pseudohyphae. A wide range of biotechnological tools for yeast manipulation are available (Madzak et al., 2004). This unconventional yeast has however not yet been exploited for the production of membrane proteins.

Thus, it would be advantageous to have expression systems that permit higher expression of proteins, particularly of membrane proteins, so that sufficient quantities can be made allowing biophysical characterization and crystallization of these proteins.

### SUMMARY

It is an object of the invention to provide cells allowing higher expression of proteins, particularly membrane proteins, and methods of expressing proteins in these cells. This is achieved by derepression of phospholipid synthesis, particularly derepression of nuclear and/or endoplasmatic reticulum (ER) phospholipid synthesis. The derepression of phospholipid synthesis genes can be achieved by elevating phosphatidic acid (PA) content at the nuclear/ER membrane. This, in turn, is achieved by inhibiting phosphatidic acid phosphatase activity and/or upregulation of diacylglycerol kinase activity, which results in expansion of the nuclear/ER membrane. Surprising in view of the significantly changed phospholipid composition as a result of increased PA levels (Han et al., 2006; Han et al., 2007) is that these membrane expansions can accommodate huge amounts of proteins, while the cells remain viable. Indeed, it is shown herein that derepression of ER phospholipid synthesis allows a 10-fold and higher increase in production of e.g. GPCRs (e.g. FIG. 4).

Thus, according to a first aspect, methods of enhancing production of proteins in a eukaryotic cell are provided, which entail that a eukaryotic cell deficient in expression and/or activity of an endogenous phosphatidic acid phosphatase, and/or overexpressing a diacylglycerol kinase is provided, wherein the cell comprises a nucleic acid sequence encoding the protein of interest and the cell is maintained in conditions suitable for expressing the protein. Afterwards, the protein or proteins of interest can then be recovered from the cell. Typically, the nucleic acid sequence encoding the protein to be produced is an exogenous nucleic acid sequence or an endogenous nucleic acid sequence under control of an exogenous promoter. The protein may be expressed constitutively or in an inducible way. Accordingly, the promoter may be a constitutive or inducible promoter.

According to particular embodiments, the endogenous phosphatidic acid phosphatase is PAH1 or a homolog thereof. According to alternative, but non-exclusive embodiments, the cell is deficient in expression and/or activity of the endogenous phosphatidic acid phosphatase through disruption of the endogenous phosphatidic acid phosphatase gene at nucleic acid level. Alternatively, the cell is deficient in expression and/or activity through an inhibitory RNA directed to the endogenous phosphatidic acid phosphatase gene transcript. Using for instance cells according to this latter embodiment, the deficiency of the expression and/or activity of the endogenous phosphatidic acid phosphatase may be inducible, which is envisaged in particular embodiments.

According to other particular embodiments, the diacylglycerol kinase that is overexpressed is DGK1 or a homolog thereof. The diacylglycerol kinase that is overexpressed may be an endogenous diacylglycerol kinase or an exogenous diacylglycerol kinase. Typically, although not necessarily, the promoter driving the diacylglycerol kinase expression is an exogenous promoter. Overexpression of the diacylglycerol kinase may be constitutive or inducible. Likewise, the promoters driving the diacylglycerol kinase expression may be constitutive or inducible promoters.

According to yet other particular embodiments, the proteins that are produced in the eukaryotic cells described herein are membrane-bound proteins. According to further particular embodiments, the protein is a receptor. According to yet further particular embodiments, the protein is a GPCR. Importantly, however, the methods may also be used to improve production of non-membrane-bound proteins, as the ER is also the place where secreted proteins are synthesized. According to specific embodiments, more than one, i.e. two or more different proteins may be produced simultaneously.

According to other envisaged embodiments, the eukaryotic cells used for protein production are yeast cells. According to even more particular embodiments, the yeast cells are methylotrophic yeast cells, such as species of the genus Hansenula (e.g. Hansenula polymorpha), species of the genus Candida (e.g. Candida boidinii) or most particularly species of the genus Pichia, such as Pichia pastoris. According to alternative embodiments, the yeast cells are of the genus Yarrowia, most particularly of the species Yarrowia lipolytica. According to very specific embodiments, the yeast cells are not from the species Saccharomyces cerevisiae or even not from the genus Saccharomyces. According to particular embodiments, the yeast cells are glyco-engineered yeast cells. Examples thereof include, but are not limited to, yeast cells that have humanized glycosylation patterns. Typically, the protein that is expressed in a yeast cell will be isolated (or possibly secreted) from the cell.

According to alternative particular embodiments, the eukaryotic cells are plant cells, particularly plant cell cultures. According to yet further alternative embodiments, the eukaryotic cells are mammalian cells, most particularly Hek293 cells, such as Hek293S cells (Reeves et al., 1996; Reeves et al., 2002).

According to specific embodiments, the methods of protein production also comprise the step of isolating the expressed protein. This typically involves recovery of the material wherein the protein is present (e.g. a cell lysate or specific fraction thereof, the medium wherein the protein is secreted) and subsequent purification of the protein. Means that may be employed to this end are known to the skilled person and include specific antibodies, tags fused to the proteins, affinity purification columns, and the like.

According to some very specific embodiments, the methods can be used to produce virus-like particles or VLPs. Indeed, the expression of viral structural proteins, such as Envelope or Capsid, can result in the self-assembly of VLPs. Thus, in these embodiments, the cells will comprise one or more nucleic acid sequences encoding the one or more proteins making up the virus-like particle, in conditions suitable for expressing the one or more proteins. According to specific embodiments where the VLP consists of more than one protein, the different proteins can also be expressed as a single polyprotein. This was demonstrated previously for different viruses (Brautigam et al., 1993; Kibenge et al., 1999).

Remarkable about VLPs is their ease of production: not only can they be produced in a variety of cell culture systems including mammalian cell lines, insect cell lines, yeast, and plant cells as well as whole plants (Santi et al., 2006), but the individual viruses they are derived from are also extremely diverse in terms of structure and include viruses that have a single capsid protein, multiple capsid proteins, and those with and without lipid envelopes (Noad and Roy, 2003). VLPs typically bud from cellular membranes (and are typically also secreted by the cell), and according to particular embodiments, the virus-like particle encompasses lipids in addition to protein(s). This is the case for VLPs derived from viruses with lipid envelopes, but also for lipoparticles (which are engineered to express membrane proteins, see e.g. Endres et al., 1997; Balliet and Bates, 1998; Willis et al., 2008; and commercially available from Integral Molecular, Philadelphia). VLPs can thus efficiently be used for production of (membrane) proteins, but also in the development of vaccines (Noad and Roy, 2003; Roy and Noad, 2008). Particular VLPs that are envisaged for vaccine production include, but are not limited to, virus-like particles based on a hepatitis virus, particularly HBV or HCV virus, on a HIV virus, on an encephalitis virus, particularly Japanese encephalitis virus, or on a Dengue virus.

According to a further aspect, eukaryotic cells are provided herein that are deficient in expression and/or activity of an endogenous phosphatidic acid phosphatase, and/or that overexpress a diacylglycerol kinase, wherein if the eukaryotic cell is a Saccharomyces cell, it comprises an exogenous nucleic acid sequence, or an endogenous nucleic acid sequence under control of an exogenous promoter, encoding a protein to be expressed and isolated from the cell. These cells can be used in the methods of enhancing protein production described herein.

According to particular embodiments, the eukaryotic cells are yeast cells. According to even more particular embodiments, the yeast cells are methylotrophic yeast cells, such as species of the genus Hansenula (e.g. Hansenula polymorpha), species of the genus Candida (e.g. Candida boidinii) or most particularly species of the genus Pichia, such as Pichia pastoris. According to alternative embodiments, the yeast cells are of the genus Yarrowia, most particularly of the species Yarrowia lipolytica. According to very specific embodiments, the yeast cells are not from the species Saccharomyces cerevisiae or even not from the genus Saccharomyces. According to particular embodiments, the yeast cells are glyco-engineered yeast cells. Examples thereof include, but are not limited to, yeast cells that have humanized glycosylation patterns.

As described for the methods above, in the eukaryotic cells the endogenous phosphatidic acid phosphatase is particularly envisaged to be PAH1 or a homolog thereof and/or the diacylglycerol kinase particularly is DGK1 or a homolog thereof.

According to specific embodiments, the eukaryotic cells further comprise an exogenous nucleic acid sequence, or an endogenous nucleic acid sequence under control of an exogenous promoter, encoding a protein to be expressed and isolated from the cell. According to further embodiments, this protein is a membrane-bound protein or a secreted protein. According to yet further specific embodiments, the protein is a receptor, more particularly a GPCR.

According to particularly envisaged embodiments, a cell culture of the eukaryotic cells as described herein is provided.

### BRIEF DESCRIPTION OF THE FIGURES

FIG. 1: Sequence of Yarrowia lipolytica PAH1. A, the nucleotide sequence of the Yarrowia lipolytica PAH1 gene with 800 bp surrounding genomic region at either side (SEQ ID NO: 1). Primers used to isolate the promoter and terminator regions of the gene are indicated. B, the amino acid sequence of the PAH1 protein of Yarrowia lipolytica is shown (SEQ ID NO: 2). The characteristic sequence of the HAD_like domain is indicated in pink, the conserved N-terminal lipin domain in blue.

FIG. 2: Sequence of Pichia pastoris PAH1. A, the nucleotide sequence of the Pichia pastoris PAH1 gene with 1000 bp surrounding genomic region at either side (SEQ ID NO: 3). Taken from IG-66 strain sequence, contig1302_{—}7261_{—}4937; the start and stop codon are indicated in green and red, respectively. B, the amino acid sequence of the PAH1 protein of Pichia pastoris is shown (SEQ ID NO: 4). The HAD_like domain is indicated in pink, the conserved N-terminal lipin domain in blue. C, protein sequence alignment of the Saccharomyces cerevisiae, Pichia pastoris, and Yarrowia lipolytica PAH1 protein.

FIG. 3: Comparison of the growth of PAH1 knock out strains versus the respective wild type strain. ΔPAH1 strains tend to show a slower growth.

FIG. 4: Expression of GPCRs in PAH1 knock out strains. The Western blot shows that the PAH1 knock out strains express higher levels of the 5HT1D receptor compared to the wild type strains. Microsomes were isolated and equal quantities of total membrane protein were loaded. At least 10-fold increase of the 5HT1D receptor can be observed. As usual, dimers and higher oligomers of the receptor are also seen. Clone 1 and 2 are clones bearing 3 copies of the 5HT1D receptor which is expressed starting from the lip2 prepro sequence of Y. lipolytica. Clone 3 (1 copy) and 4 (2 copies) express the receptor with its own signal sequence.

FIG. 5: TEM picture of Yarrowia lipolytica with disruption in the PAH1 gene. The magnification shows the massive proliferation of tightly stacked membranes, continuous with the ER.

FIG. 6: sequences of yeast DGK1. A, D: nucleotide (SEQ ID NO: 5) and amino acid (SEQ ID NO: 6) sequence of Yarrowia lipolytica DGK1, respectively. B, E: nucleotide (SEQ ID NO: 7) and amino acid (SEQ ID NO: 8) sequence of Pichia pastoris DGK1, respectively. C, F: nucleotide (SEQ ID NO: 9) and amino acid (SEQ ID NO: 10) sequence of Saccharomyces cerevisiae DGK1, respectively. G, protein sequence alignment of the Pichia pastoris, Saccharomyces cerevisiae and Yarrowia lipolytica DGK1 protein.

FIG. 7: expression of GPCRs in DGK1 overexpression strains. Western blotting of 3 Pichia pastoris strains overexpressing the dgk1p together with the A2A receptor. Equal amounts of protein were loaded on the gel. Blot shows a higher expression level for the dgk1 overexpressing strains (clone 1-3) compared to the wild type strain (WT).

FIG. 8: Comparison of the growth of a DGK1 overexpressing strain versus the respective wild type strain. Growth rates are comparable.

FIG. 9: TEM picture of Pichia pastoris strains overexpressing the DGK1 gene. The arrows show the membranes induced by the DGK1p overexpression.

FIG. 10 shows the cloning strategy used for disruption of the PAH1 gene of Pichia pastoris.

FIG. 11: Growth analysis of the Pichia pastoris strain with a disrupted PAH1 gene and the corresponding wild type strain. Cells are grown to saturation, diluted to 2.10e⁷ cells/ml and serial dilutions (1:10) of the cells are spotted (5 μl). Top panel: growth at 30° C.; bottom panel: growth at 37° C.

FIG. 12: TEM picture of Pichia pastoris strains with disruption in the PAH1 gene. The arrows show the membranes induced by the lack of PAH1.

FIG. 13: Electron Microscopy Pictures of ΔPAH1 P. pastoris Cells on Glucose and Oleic Acid.

ΔPAH1 cells grown on glucose show extra ER membrane which are mostly internalized in vacuoles. The PAH1 KO strain grown on oleic acid has a much more pronounced increase in ER membrane which are not present in vacuoles. A strategy to increase the effect of the PAH1 KO on membrane protein expression might be to express the receptor from a fatty acid inducible promoter.

FIG. 14: Expression and functionality of GPCRs in PAH1 knock out and DGK1 overexpression strains of Pichia pastoris. A, Comparison of the expression levels of the A2AR in the WT, PAH1 and DGK1 strain in Pichia pastoris. B, Radioligand binding studies on membranes of P. pastoris cells expressing the A2AR. 5 μg of total membrane protein was incubated with different concentrations of the A2AR antagonist ³[H]ZM241385 in 500 μl binding buffer. Non-specific binding was determined in the presence of 10 mM theophylline. Bound and free ligand were separated on Whatmann GF/B filters using a Brandel harvester. Data were analyzed with the software KaleidaGraph from Synergy Software.

FIG. 15: Expression and functionality of GPCRs in a PAH1 knock out strain of Yarrowia lipolytica. A, Western blot analysis of the Adenosine A2A receptor in Yarrowia lipolytica. Each lane corresponds with 5 μg total membrane protein. Lane 1 shows the expression level of the wild type strain and lane 2 the expression level for the knock out strain. A clear increase in expression level is seen in the PAH1 knock out strain. B, saturation binding assay on 5 μg total membrane protein from Y. lipolytica cells expressing the A2A receptor. Conditions are the same as for FIG. 14B.

FIG. 16: Growth analysis of a Yarrowia lipolytica PAH1 knock out strain on glucose and oleic acid. Yeast cells were grown overnight in YPD and than inoculated at an OD 0.1 in YPO (1% yeast extract/2% peptone/2% oleic acid) or YPD. Cells were incubated at 28° C. with continuous shaking at 250 rpm. At the indicated time points samples were taken for absorbance measurements. Data points represent the average of three measurements. A, growth on YPD for 27 hours. B, growth on oleic acid 55 h.

FIG. 17: Electron Microscopy Pictures Taken at the End of A2A Receptor Induction.

Yarrowia lipolytica PAH1 knock out strains show a clear increase in ER membrane surface. Especially the strains grown on oleic acid have multiple layers of induced ER membrane. The PAH1 strain grown on oleic acid has no or little lipid droplets compared to the wild type strain.

FIG. 18: A. Western Blot Analysis of A2A Receptor Overexpressing Strains

Lane 1 the EV strain not expressing the A2A receptor. Lane 2 represents the strain expressing the A2A receptor from the POX2 promoter. Lane 3 is the PAH1 KO strain. Lanes 4 and 5 show the A2A expression from the hp4d promoter in a wild type and KO strain respectively.

B. Ligand Binding Tests on the A2A Receptor

Panels on the left show ligand binding tests of the A2A receptor expressed from the oleic acid inducible promoter POX2. Graphs are shown for 2 independent experiments. No binding was obtained for the wild type strain expressing the A2A receptor despite the fact that expression was observed on western blot (FIG. 18 A lane 2). Knocking out the PAH1 gene in this strain increases the expression which is reflected in binding of the A2A receptor ligand ZM241385.

Panels on the right show ligand binding results of the A2A receptor expressed from the hp4d promoter. Again graphs show 2 independent experiments. The PAH1 strain shows more receptor binding than the wild type strain.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

A “eukaryotic cell” as used herein is a cell containing a nucleus and an endoplasmatic reticulum or ER, which is involved in protein transport and maturation.

The term “endogenous” as used herein, refers to substances (e.g. genes) originating from within an organism, tissue, or cell. Analogously, “exogenous” as used herein is any material originated outside of an organism, tissue, or cell, but that is present (and typically can become active) in that organism, tissue, or cell.

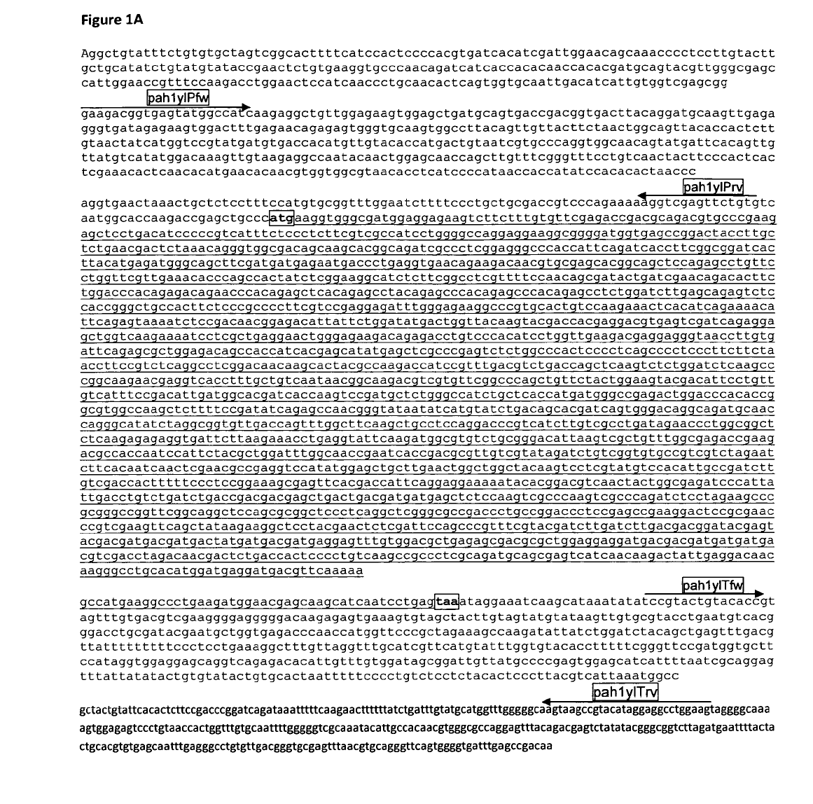

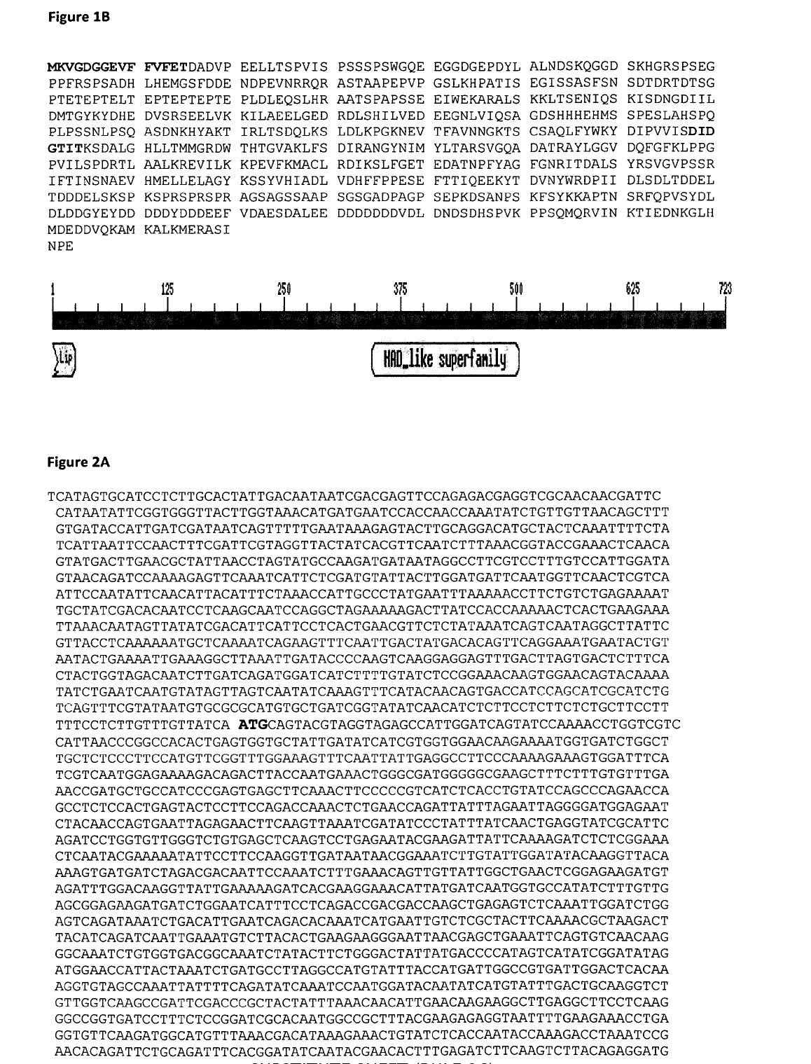

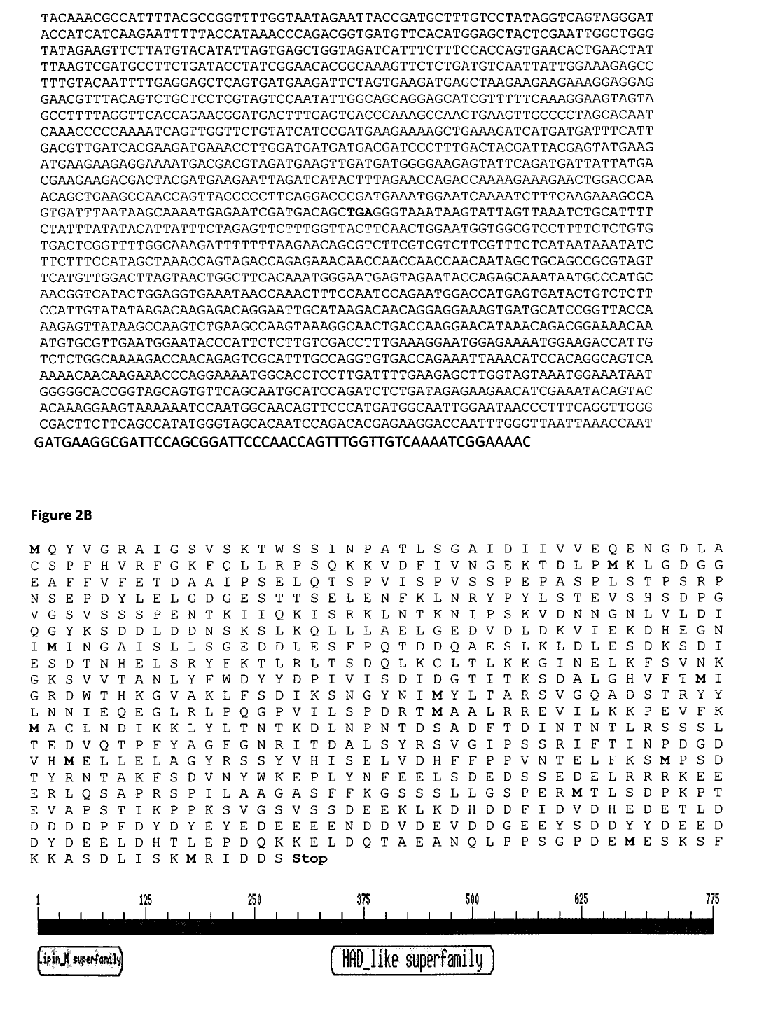

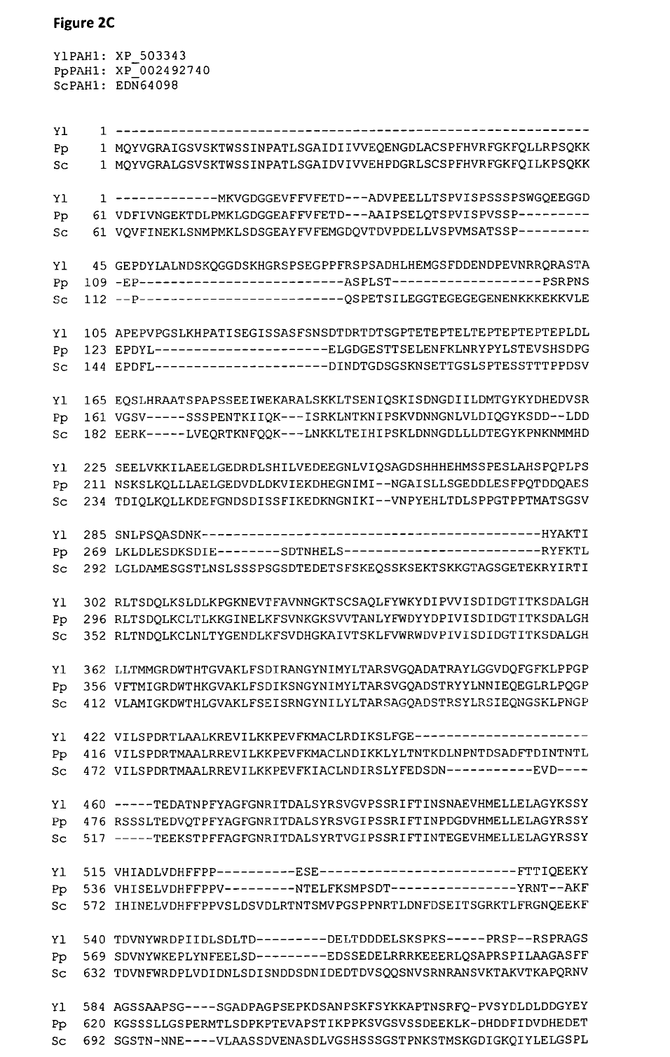

The term “phosphatidic acid phosphatase” or “PAP” is used herein to designate an enzyme catalyzing the dephosphorylation of phosphatidic acid (EC 3.1.3.4), thereby yielding diacylglycerol (DAG) and Pᵢ. Most particularly, PAP is specific for PA and requires Mg²⁺ for activity, to distinguish from lipid phosphate phosphatase, also designated as PAP2, which is not specific for phosphatidic acid and does not require Mg²⁺ for activity (although it helps in reaching maximal activity) (Carman and Han, 2006). The term “PAH1” as used herein refers to the yeast PAP enzyme and the encoding gene (Gene ID: 855201 in Saccharomyces cerevisiae; gene and protein sequences of the Yarrowia lipolytica and Pichia pastoris PAH1 are shown in FIGS. 1 and 2, including an alignment with the Saccharomyces cerevisiae PAH1 protein), sometimes also indicated as SMP2 (Santos-Rosa et al., 2005; Han et al., 2006).

A “PAH1 homolog” as used throughout the application refers to genes and proteins in species other than yeast homologous to PAH1 and having PAP activity. Homology is expressed as percentage sequence identity (for nucleic acids and amino acids) and/or as percentage sequence similarity (for amino acids). Preferably, homologous sequences show at least 50%, at least 60%, at least 70%, at least 80%, at least 90%, at least 95% or at least 99% sequence identity at nucleic acid level or sequence identity or similarity at amino acid level. Algorithms to determine sequence identity or similarity by sequence alignment are known to the person skilled in the art and include for instance the BLAST program. Alternatively, homologs can be identified using the HomoloGene database (NCBI) or other specialized databases such as for instance HOGENOM or HOMOLENS (Penel et al., 2009). Examples of PAH1 homologs include, but are not limited to, lipins in mammalians and some other vertebrates (encoded by Lpin1, Lpin2, and Lpin3; Gene ID: 23175, 9663, and 64900 in humans and 14245, 64898 and 64899 in mice, respectively), ned1 in Schizosaccharomyces (GeneID: 2542274), CG8709 in Drosophila (GeneID: 35790), AgaP_AGAP007636 in Anopheles (GeneID: 1269590), and AT3G09560 (GeneID: 820113) and AT5G42870 (GeneID: 834298) in Arabidopsis thaliana. A particularly envisaged PAH1 homolog is lipin-1. Typical of these PAH1 homologs is that they possess a NLIP domain with a conserved glycine residue at the N-terminus and a HAD-like domain with conserved aspartate residues in the catalytic sequence DIDGT (SEQ ID NO: 11) (Péterfy et al., 2001; Han et al., 2007; Carman and Han, 2009).

The term “diacylglycerol kinase”, “DAGK” or “DGK” as used herein refers to an enzyme catalyzing the reverse reaction as a phosphatidic acid phosphatase, i.e. the phosphorylation of DAG to obtain phosphatidic acid (EC 2.7.1.107 for the ATP-dependent DGK; in yeast, the enzyme is CTP-dependent (Han et al., 2008a, 2008b) and EC 2.7.1.n5 has been proposed as nomenclature in the Uniprot database).

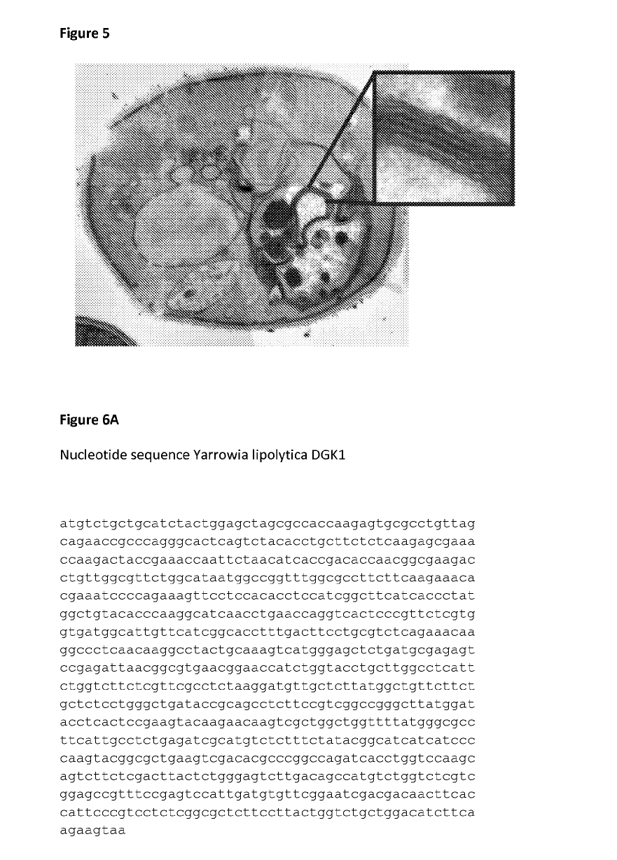

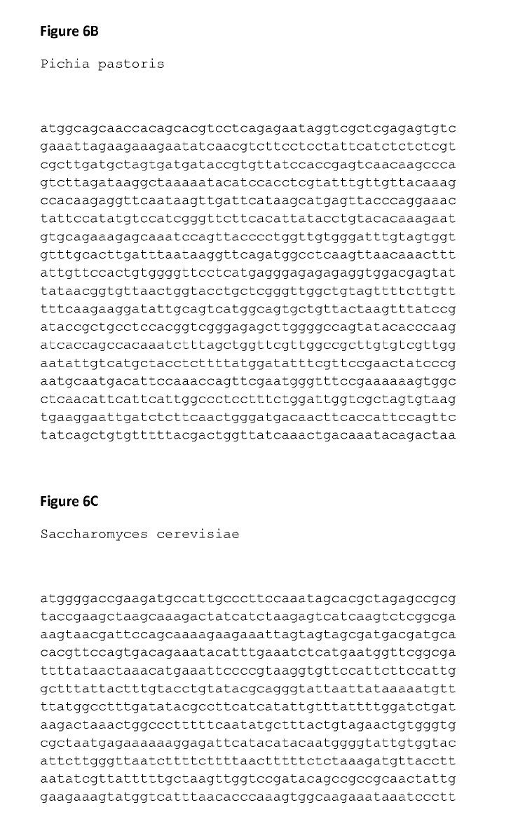

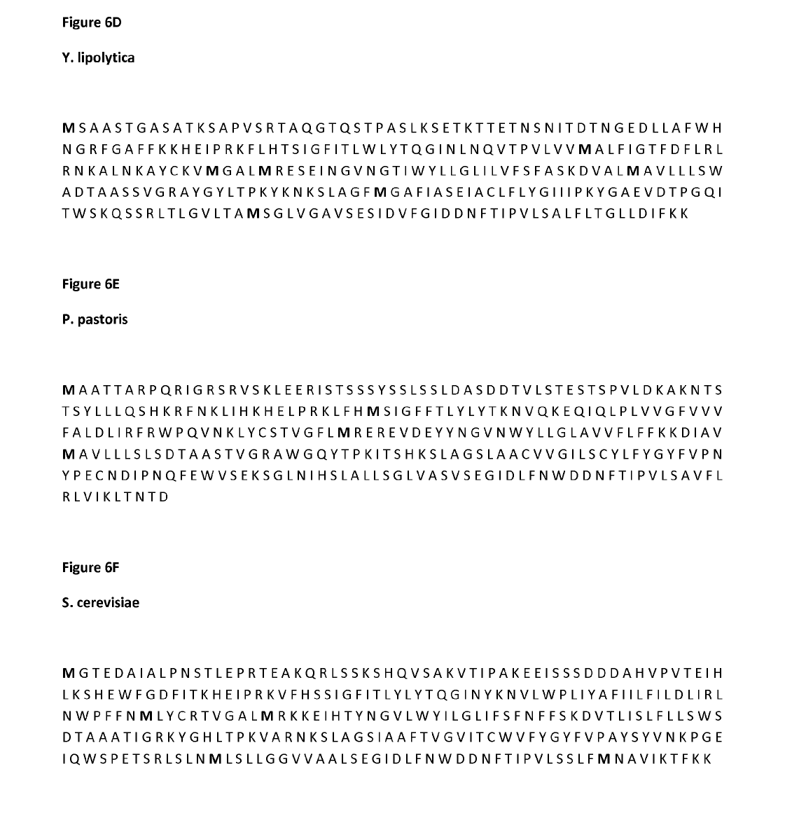

The term “DGK1” as used herein refers to the yeast DGK enzyme and the encoding gene (GeneID: 854488 in Saccharomyces cerevisiae; Gene ID: 8199357 in Pichia Pastoris and Gene ID: 2909033 for Yarrowia lipolytica. The gene and protein sequences of these DGK1s are also shown in FIG. 6), sometimes also indicated as HSD1.

A “DGK1 homolog” as used throughout the application refers to genes and proteins in species other than yeast homologous to DGK1 and having diacylglycerol kinase activity. Homology is as detailed above. DGK1 homologs are found throughout the eukaryotes, from yeast over plants (Katagiri et al., 1996; Vaultier et al., 2008) to C. elegans (Jose and Koelle, 2005) and mammalian cells (Sakane et al., 2007). Typically, DGK1 in yeast uses CTP as the phosphate donor in its reaction (Han et al., 2008b) while DGK1 homologs in e.g. mammalian cells use ATP instead of CTP (Sakane et al., 2007).

The term “inducible promoter” as used herein refers to a promoter that can be switched ‘on’ or ‘off’ (thereby regulating gene transcription) in response to external stimuli such as, but not limited to, temperature, pH, certain nutrients, specific cellular signals, etcetera. It is used to distinguish between a “constitutive promoter”, by which a promoter is meant that is continuously switched ‘on’, i.e. from which gene transcription is constitutively active.

A “glyco-engineered yeast” as used herein is a yeast wherein the naturally occurring modifications on glycoproteins have been altered by genetic engineering of enzymes involved in the glycosylation pathway. A “glycoprotein” is a protein that carries at least one oligosaccharide chain. Typical monosaccharides that may be included in an oligosaccharide chain of a glycoprotein include, but are not limited to, glucose (Glu), galactose (Gal), mannose (Man), fucose (Fuc), N-acetylneuraminic acid (NeuAc) or another sialic acid, N-acetylglucosamine (GlcNAc), N-acetylgalactosamine (GalNAc), xylose (Xyl) and derivatives thereof (e.g. phosphoderivatives). Sugar chains may be branched or not, and may comprise one or more types of oligosaccharide. In general, sugar chains in N-linked glycosylation may be divided in three types: high-mannose, complex and hybrid type glycosylation. These terms are well known to the skilled person and defined in the literature. Briefly, high-mannose type glycosylation typically refers to oligosaccharide chains comprising two N-acetylglucosamines with (possibly many) mannose and/or mannosylphosphate residues (but typically no other monosaccharides).

Complex glycosylation typically refers to structures with typically one, two or more (e.g. up to six) outer branches with a sialyllactosamine sequence, most often linked to an inner core structure Man3GlcNAc2. For instance, a complex N-glycan may have at least one branch, or at least two, of alternating GlcNAc and galactose (Gal) residues that may terminate in a variety of oligosaccharides but typically will not terminate with a mannose residue.

Hybrid type glycosylation covers the intermediate forms, i.e. those glycosylated proteins carrying both mannose and non-mannose residues in addition to the two N-acetylglucosamine residues. In contrast to complex glycosylation, at least one branch of hybrid type glycosylation structures ends in a mannose residue.

Typically, but not necessarily, natural yeast will have a high mannose type glycosylation, and it can be glyco-engineered in part or in whole towards hybrid glycosylation or even complex glycosylation (to more closely resemble glycosylation found in mammalian cells). Examples of glyco-engineered yeast strains are known in the art (e.g. Wildt and Gerngross, 2005; Jacobs and Callewaert, 2009).

A “virus-like particle” or “VLP” as used herein consist of proteins that form a virus's outer shell and the surface proteins, without the genetic material required for replication. It thus forms a genetically engineered spherical protein envelope derived from a virus that does not contain viral genetic material and cannot replicate but can elicit an immune response and thus be used for vaccine purposes. VLPs have been produced from components of a wide variety of virus families including Parvoviridae (e.g. adeno-associated virus), Retroviridae (e.g. HIV), and Flaviviridae (e.g. Hepatitis C virus). Although VLPs may be entirely made up of proteins, a particular class of VLPs are lipid-containing VLPs (e.g. derived from viruses with a lipid envelope, such as influenza or herpes viruses) or lipoparticles (U.S. Pat. No. 7,763,258), which contain lipids in their outer shell, which makes them particularly suitable for the expression of membrane proteins.

It is an object of the invention to provide cells or cellular systems (cultures, organisms) that can express high concentrations of proteins, particularly membrane proteins. Also, methods are provided that use such cells or cellular systems to produce higher amounts of such proteins than is feasible with existing methods.

According to a first aspect, eukaryotic cells are provided deficient in expression and/or activity of an endogenous phosphatidic acid phosphatase, and/or overexpressing a diacylglycerol kinase. It is particularly envisaged that in some embodiments, these cells are not of the species Saccharomyces cerevisiae. The cells will further typically contain a nucleic acid sequence encoding a protein to be expressed in said cell. Most particularly, the nucleic acid sequence is an exogenous sequence or an endogenous sequence under control of an exogenous promoter. In these embodiments, the cells may also be of the species Saccharomyces cerevisiae.

Accordingly, methods of enhancing protein production in such eukaryotic cells are provided. These methods entail that a eukaryotic cell deficient in expression and/or activity of an endogenous phosphatidic acid phosphatase, and/or overexpressing a diacylglycerol kinase is provided, wherein the cell comprises a nucleic acid sequence encoding the protein of interest and the cell is maintained in conditions suitable for expressing the protein. Particularly where the eukaryotic cell is from the species Saccharomyces cerevisiae, the nucleic acid sequence encoding the protein of interest is an exogenous sequence or an endogenous sequence under control of an exogenous promoter. This is envisaged for other species as well. The expressed protein may further optionally be isolated and/or purified.

The nature of the eukaryotic cells, both as such and as used in the methods provided herein, can be very varied, since all eukaryotic cells have an endoplasmatic reticulum and it is through expansion of this membrane that protein production is increased. Also, phosphatidic acid phosphatases and diacylglycerol kinases occur in all kinds of eukaryotic cells, and it has been shown that their function is evolutionarily conserved from unicellular eukaryotes to mammals (Grimsey et al., 2008). In this regard, it should be stressed that the technical effect of PAP inhibition is identical to that of increasing DGK activity, since the enzymes catalyze opposite directions of the same reaction.

It is particularly envisaged that the eukaryotic cells used are eukaryotic cells that are normally used as expression systems, to take further advantage of optimized protein production. Examples of eukaryotic cells that are used for protein production include, but are not limited to, yeast cells (e.g. Pichia, Hansenula, Yarrowia), insect cells (e.g. SF-9, SF-21, and High-Five cells), mammalian cells (e.g. Hek293, COS, CHO cells), plant cell cultures (e.g. Nicotiana tabacum, Oryza sativa, soy bean or tomato cultures, see for instance Hellwig et al., 2004; Huang et al., 2009), or even whole plants. The cells may thus be provided as such, as a eukaryotic cell culture, or even as an organism (i.e. a non-human organism). According to particular embodiments, however, the organism is not a mouse, or not even a mammal.

It is particularly envisaged that the eukaryotic cells are yeast cells, as these are very amenable to protein production and are robust expression systems. According to even more particular embodiments, the yeast cells are methylotrophic yeast cells, such as species of the genus Hansenula (e.g. Hansenula polymorpha), species of the genus Candida (e.g. Candida boidinii) or most particularly species of the genus Pichia, such as Pichia pastoris. According to alternative embodiments, the yeast cells are of the genus Yarrowia, most particularly of the species Yarrowia lipolytica. According to very specific embodiments, the yeast cells are not from the species Saccharomyces cerevisiae or even not from the genus Saccharomyces. According to particular embodiments, the yeast cells are glyco-engineered yeast cells. Examples thereof include, but are not limited to, yeast cells that have humanized glycosylation patterns. Typically, the protein that is expressed in a yeast cell will be isolated (or possibly secreted) from the cell.

According to alternative particular embodiments, the eukaryotic cells are plant cells, particularly plant cell cultures. According to yet further alternative embodiments, the eukaryotic cells are mammalian cells, most particularly Hek293 cells, such as Hek293S cells.

To make a cell deficient in expression and/or activity of an endogenous phosphatidic acid phosphatase, several strategies can be used, and the nature of the strategy is not vital to the invention, as long as it results in diminishing PAP activity to the extent that the endoplasmatic reticulum is expanded in the cell. Cells can be made deficient for PAP at the genetic level, e.g. by deleting, mutating, replacing or otherwise disrupting the (endogenous) gene encoding PAP. Alternatively, one can interfere with transcription from the PAP gene, or remove or inhibit the transcribed (nucleic acid, mRNA) or translated (amino acid, protein) gene products. This may for instance be achieved through siRNA inhibition of the PAP mRNA. Also morpholinos, miRNAs, shRNA, LNA, small molecule inhibition or similar technologies may be used, as the skilled person will be aware of. The PAP protein can for instance be inhibited using inhibitory antibodies, antibody fragments, scFv, Fc or nanobodies, small molecules or peptides.

Interestingly, PAP activity may also be inhibited without directly interfering with PAP expression products. For instance, in yeast it has been shown that the loss of the dephosphorylated form of the yeast PAP enzyme PAH1 by deletion of Nem1 and/or Spo7, which form a complex that dephosphorylates PAH1, results in the same phenotype as deletion of PAH1 (Siniossoglou et al, 1998; Santos-Rosa et al., 2005). Likewise, increased phosphorylation of Lipin 1 and 2 inhibits their PA phosphatase activity (Grimsey et al., 2008). Thus, a cell may be made deficient in PAP activity by increasing PAP phosphorylation (or blocking PAP dephosphorylation).

As will be clear to those of skill in the art, deficiency of PAP expression and/or activity may both be constitutive (e.g. genetic deletion) or inducible (e.g. small molecule inhibition).

Typically, the endogenous phosphatidic acid phosphatase is PAH1 or a homolog thereof.

The eukaryotic cells provided herein may, either solely or in addition to PAP deficiency, overexpress a diacylglycerol kinase. This may be endogenous DGK that is overexpressed, for instance by means of an exogenous promoter. The exogenous promoter may be constitutive or inducible, but typically will be a stronger promoter than the endogenous promoter, to ensure overexpression of DGK. Alternatively, an exogenous diacylglycerol kinase is overexpressed, i.e. the eukaryotic cell is genetically engineered so as to express a DGK that it does not normally express. The exogenous DGK may for instance also be a non-naturally occurring DGK, such as for instance a functional fragment of a diacylglycerol kinase. A fragment is considered functional if it retains the capability to catalyze the phosphorylation reaction of DAG to obtain phosphatidic acid. As for endogenous DGK, the exogenous DGK may also be under control of a constitutive or inducible promoter. The nature of the promoter is not vital to the invention and will typically depend on the expression system (cell type) used and/or on the amount of protein that is needed or feasible. According to particular embodiments, the diacylglycerol kinase that is overexpressed is DGK1 or a homolog thereof.

Particularly envisaged are cells that combine a deficiency in endogenous PAP with overexpression of a DGK, for even higher production of proteins, although the effect is not necessarily additive. Note that the cells described herein that are characterized by significant membrane expansion (by having a PAP deficiency and/or overexpressing a DGK) may be further engineered for increased protein expression. A non-limiting example thereof is overexpression of HAC1 (Guerfal et al., 2010), but other modifications are also known in the art. Alternatively or additionally, the cells may be further engineered to perform eukaryotic post-translational modifications (e.g. De Pourcq et al., 2010).

The proteins that are produced in the cells described herein will typically be encoded by an exogenous nucleic acid sequence or an endogenous nucleic acid sequence under control of an exogenous promoter, i.e. the cells are engineered to express the protein of interest. The protein may be expressed constitutively or in an inducible way. Accordingly, the promoter may be a constitutive or inducible promoter.

According to particular embodiments, the proteins that are produced in the eukaryotic cells described herein are membrane-bound proteins. According to further particular embodiments, the protein is a receptor. According to yet further particular embodiments, the protein is a GPCR. Examples of GPCRs envisaged for production using the methods provided herein include, but are not limited to, Calcium-sensing receptor, CXCR4, endothelin receptor B, follicle stimulating hormone receptor, N-formyl peptide receptor, frizzled receptor (FZD4), gonadotropin-releasing hormone receptor, GPR54, GPR56, Kaposi's sarcoma constitutively active GPCR, melanocortin 4 receptor, rhodopsin, vasopressin receptor, β1 adrenergic receptor, β2 adrenergic receptor, β3 adrenergic receptor, a adrenergic receptors, CCR2, CCR5, dopamine receptor, dopamine receptor 2, dopamine receptor 3, muscarinic receptor subtype 3, GPR154, P2Y₁₂, 5-HT receptors, angiotensin II receptors, adenosine A2AR, substance PNK1R, serotonin receptors (e.g. 5HT1D), histamine receptors (e.g. HH1R), opioid receptors (e.g. OPRK), and parathyroid hormone receptor 1 (see e.g. Insel et al., 2007 for a review of some of these).

The methods for GPCR expression provided herein can also be combined with known improvements for GPCR expression that typically involve production of GPCR variants. Examples thereof include, but are not limited to, the use of a signal sequence specific to the species of eukaryotic cell used rather than the GPCR-specific signal sequence, the use of a C-truncated GPCR versus the use of an intact GPCR, the use of a GPCR with a sequence insertion (e.g. a T4 lysozyme coding sequence) in the 3^{rd} loop, and so on. Of course, these different variations can further be combined with each other.

Apart from receptors, the cells and methods may also be used to improve production of secreted proteins, which are also synthesized in the ER. The increased membrane surface (and enclosed volume) indeed allows for more proteins to be synthesized and secreted.

According to specific embodiments, the produced proteins are functional, for instance produced receptors remain capable of ligand binding and signal transduction.

According to specific embodiments, more than one, i.e. two or more different proteins may be produced simultaneously. The proteins may all be membrane-bound, all be secreted proteins or a mixture thereof. When more than one protein is produced, care will typically be taken that they can be recovered easily either separately or together. In a specific embodiment, even higher production is achieved by expressing multiple copies of the protein to be expressed, e.g. as a polyprotein.

A special case of proteins that are envisaged to be produced together are proteins that form supramolecular entities. One such example are structural proteins, e.g. structural proteins making up a virus-like particle (VLP). VLPs mimic the overall structure of virus particles without any requirement that they contain infectious genetic material, and thus are ideally suited as a highly effective type of subunit vaccines. Indeed, many VLPs lack the DNA or RNA genome of the virus altogether, but have the authentic conformation of viral capsid proteins seen with attenuated virus vaccines, without any of the risks associated with virus replication or inactivation. VLP preparations are all based on the observation that expression of the capsid proteins of many viruses leads to the spontaneous assembly of particles that are structurally similar to authentic virus (Miyanohara et al., 1986; Delchambre et al., 1989; Gheysen et al., 1989; French et al., 1990). Moreover, VLPs have been successfully produced in many expression systems, such as yeast and insect cell systems. Interestingly, many VLPs bud from the internal (ER-derived) membranes. As it is feasible to make VLPs from viruses with lipid envelopes which are derived from the host cell (such as influenza, HIV and HCV; Roy and Noad, 2008), the enhanced membrane formation seen in the cells provided herein can serve to increase VLP production in the cells. This holds true for VLPs budding from ER-derived membranes, but also for VLPs budding from the plasma membrane, as membrane proteins are synthesized in ER prior to being transported to the plasma membrane.

In those embodiments where the expression of viral structural proteins, such as Envelope or Capsid, is envisaged for self-assembly of VLPs, the cells will comprise one or more nucleic acid sequences encoding the one or more proteins making up the virus-like particle, in conditions suitable for expressing the one or more proteins. According to specific embodiments where the VLP consists of more than one protein, the different proteins can also be expressed as a single polyprotein (Brautigam et al., 1993; Kibenge et al., 1999). As mentioned, VLPs can be generated for a whole variety of viruses, infecting humans, animals or even plants. These include, but are not limited to, HIV, respiratory syncytial virus (RSV), hepatitis B virus, hepatitis C virus, hepatitis E virus, Epstein Barr virus, HPV, measles virus, influenza virus, influenza A virus, Norwalk and Norwalk-like virus, feline calicivirus, porcine parvovirus, mink enteritits parvovirus, canine parvovirus, B19, AAV, chicken anemia virus, porcine circovirus, SV40, JC virus, murine polyomavirus, polio virus, bluetongue virus, rotavirus, SIV, FIV, Visna virus, feline leukemia virus, BLV, Rous sarcoma virus, Newcastle disease virus, SARS coronavirus, Hantaan virus, infectious Bursal disease virus, Dengue virus, and encephalitis virus (see e.g. Noad and Roy, 2003; Roy and Noad, 2008; Sugrue et al., 1997; Falcon et al., 1999; Liu et al., 2010).

During or after the protein production in the eukaryotic cells, the protein or proteins of interest can be recovered from the cells. Accordingly, the methods of protein production may optionally also comprise the step of isolating the expressed protein. This typically involves recovery of the material wherein the protein is present (e.g. a cell lysate or specific fraction thereof, the medium wherein the protein is secreted) and subsequent purification of the protein. Means that may be employed to this end are known to the skilled person and include specific antibodies, tags fused to the proteins, affinity purification columns, and the like.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

### Identification and Knock-Out of PAH1 in Yarrowia lipolytica

It is shown that the knock out of a single gene PAH1 in the yeast Yarrowia lipolytica can lead up to a 10-fold higher expression of eukaryotic membrane proteins. PAH1 has been identified in S. cerevisiae as a protein that couples phospholipid synthesis to growth of the nuclear/endoplasmic reticulum membrane (Santos-Rosa et al., 2005). Mutants defective in the PAH1 gene show a massive nuclear/ER membrane expansion.

The sequence of the PAH1 gene in Yarrowia lipolytica was found through a homology search of the Saccharomyces cerevisiae PAH1 (GeneID: 855201) gene against the genome of Y. lipolytica using the Basic Local Alignment search Tool (Blast) from ncbi. The blast revealed the protein YALI0D27016g (FIG. 1), which shows some similarities with the nuclear elongation and deformation protein 1 from Schizosaccharomyces pombe. Analysis of the sequence shows the presence of the conserved haloacid dehalogenase (HAD)-like domain in the middle of the protein sequence which contains a D×D×T motif (Han et al., 2007). Also the terminal N-terminal lipin domain, characteristic for the PAH1 gene, could be identified (FIG. 1B).

A PAH1 knock out construct was generated on leu2 as selection marker and transformed to 4 clones expressing a different amount of 5HT1D expression cassettes. The gene construction strategy used to knock out the PAH1 gene is described in the material and methods section. The gene was disrupted by double homologous recombination after transformation of the linearized knock out fragment to the respective strains. Clones phototrophic for leucine were screened with PCR on genomic DNA with the primers pah1ylPfw07-010: 5′GCGGCCGCGAAGACGGTGAGTATGGCCATC 3′ (SEQ ID NO: 12) and pah1ylTrv07-007: 5′ GCGGCCGCCCAAACCATGCATACAAATCAG 3′ (SEQ ID NO: 13). Positive identified clones by PCR were confirmed by Southern blot. Knock outs do not show the wild type fragment of 758 bp but a band of 1253 bp. Random integrants show bands of both sizes.

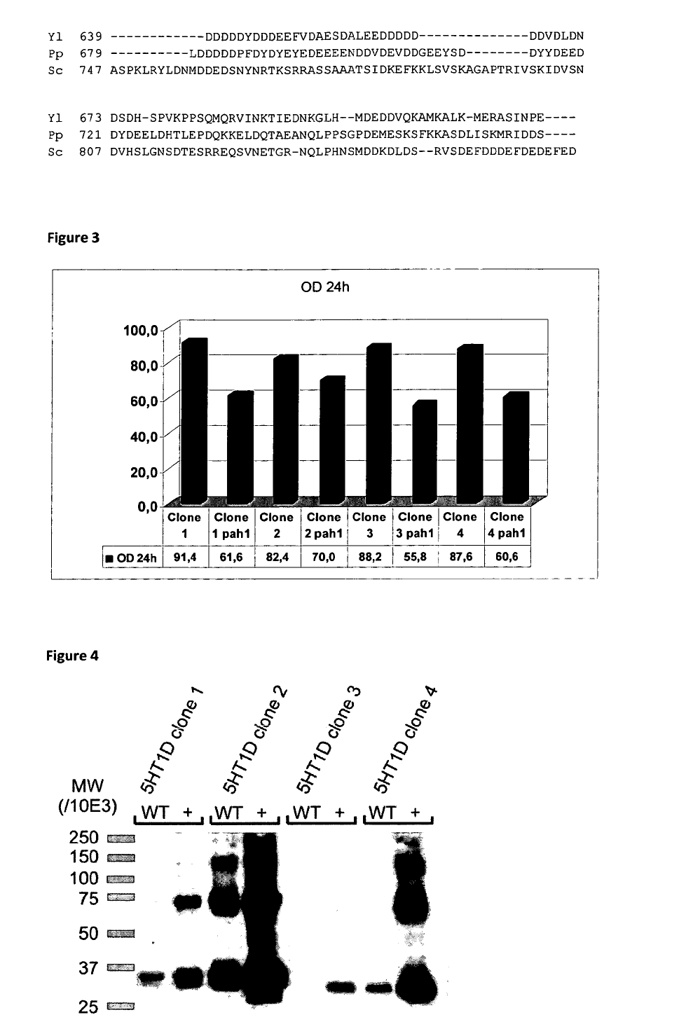

To analyze the expression levels of the 5HT1D receptor of the knock out strains versus the wild type strains, an expression experiment was performed. Cells were induced on oleic acid for 24 hours and then harvested. Before and after induction the optical density was measured. It could clearly be seen that the PAH1 knock out strains show a slower growth compared to the WT strains (FIG. 3).

To evaluate the expression levels of the receptor, cells were broken with glass beads and membrane fractions were isolated. A BCA protein assay was done to determine the amount of total membrane protein and a same amount of the proteins was loaded on a 12% SDS-PAGE gel. After separation of the proteins, proteins were blotted on to a nitrocellulose membrane (PerkinElmer). Immunoblotting shows an at least ten fold higher expression of the receptor compared to the wild type strains (FIG. 4).

Electron microscopy was performed in order to obtain a phenotypical analysis of the PAH1 knock out strains. On the TEM pictures it could be seen that the knock out strain shows a region of stacked membranes which are continuous with the outer nuclear membrane and hence the ER membrane (FIG. 5). These stacked membranes were also reported in the S. cerevisiae knock out strain (Santos-Rosa et al., 2005).

### Example 2

### Isolation and Cloning of the Diacylglycerol Kinase 1 (DGK1) of Pichia pastoris

Recently an enzyme counteracting the phosphaptase activity of PAH1 was identified in S. cerevisiae (Han et al., 2008a and b). It is reported that overexpression of DGK1 leads to an accumulation of PA and consequently expansion of the nuclear/ER membrane as seen in the Δpah1 mutant. Unlike hydroxymethylglutaryl-Co-A and cytochrome b5 which induce membrane expansion in the form of karmellae also when the catalytically inactive enzymes are overexpressed, membrane expansion is only observed when active dgk1p is overexpressed (Han et al., 2008b).

The DGK1 sequence of P. pastoris was found through a homology search of the S. cerevisiae DGK1 against the P. pastoris genome. Primers were ordered to isolate the DGK1 from the genome (DGK1pp09-007 (5′-AAAAACAACTAATTATTCGAAATGGCAGCAACCACAGCACGTCC-3′ (SEQ ID NO: 14)) and DGK1pp09-008 (5′-AAGGCGAATTAATTCGCGGCCGCTTAGTCTGTATTTGTCAGTTTG-3′ (SEQ ID NO: 15))). After isolation the gene was cloned in the pPIChyg vector resulting in the plasmid pPIChygDGK1 using the CloneEZ kit from GenScript. The vector was linearized in the AOX1 promotor with the restriction enzyme Pme1 and transformed via electroporation to a P. pastoris strain expressing the Adenosine A2A receptor under control of the AOX1 promotor. Integration of the plasmid was confirmed through PCR on genomic DNA.

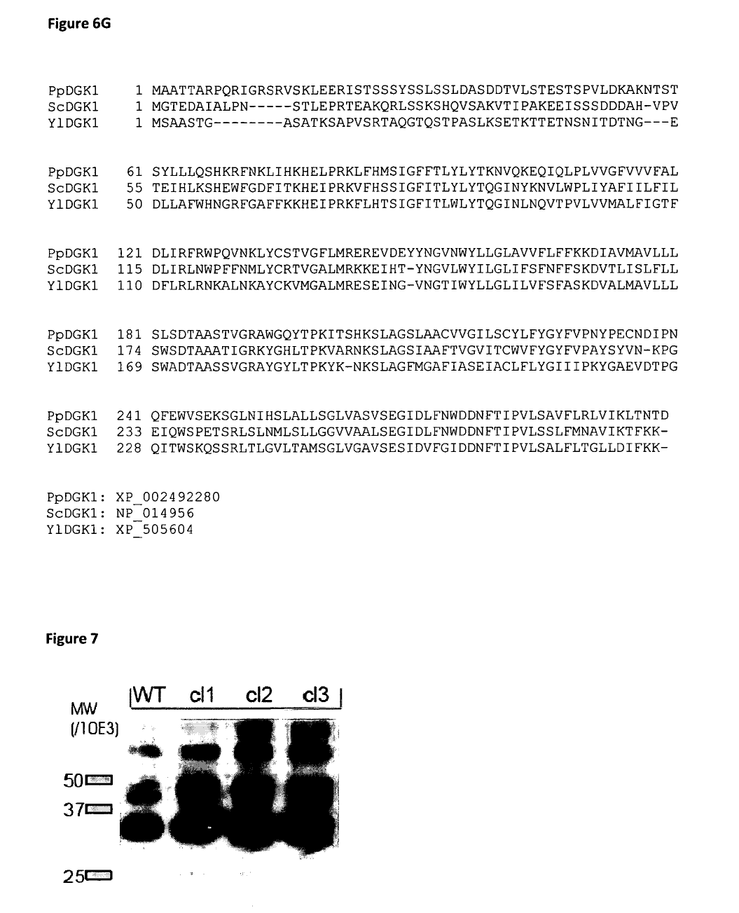

Positive clones were evaluated on the expression of the A2A receptor. Clones were grown for 48 h on BMGY, washed once with BMY and then resuspended in BMMY (1% methanol). Methanol was added every 12 hours to sustain protein induction. After induction cells were harvested and evaluated for receptor expression through western blot using the Rho1D4 tag present on the protein. From the Western Blot it is seen that the dgk1p overexpressing strains show a higher expression of the receptor (FIG. 7).

Growth of the dgk1p Overexpressing Strain.

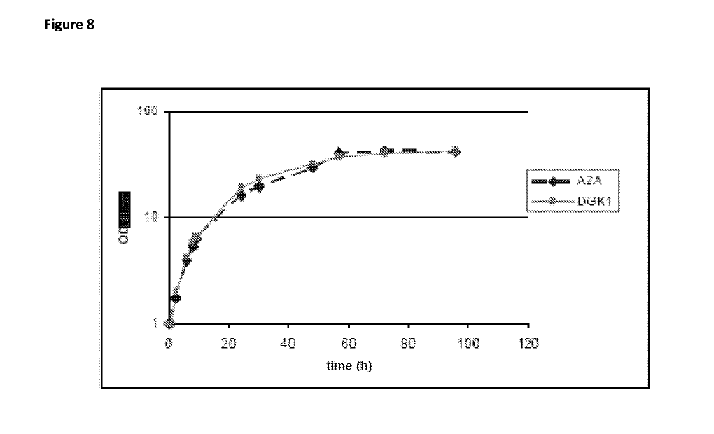

As it is known that the S. cerevisae Δpah1 show a growth defect (Santos-Rosa et al., 2005), we evaluated if the dgk1p overexpressing strain also shows slower growth at 30° C. A preculture was grown in BMGY, OD600 was measured and cells were diluted to an OD600 of 1 in BMMY. However, no slower growth was observed (FIG. 8).

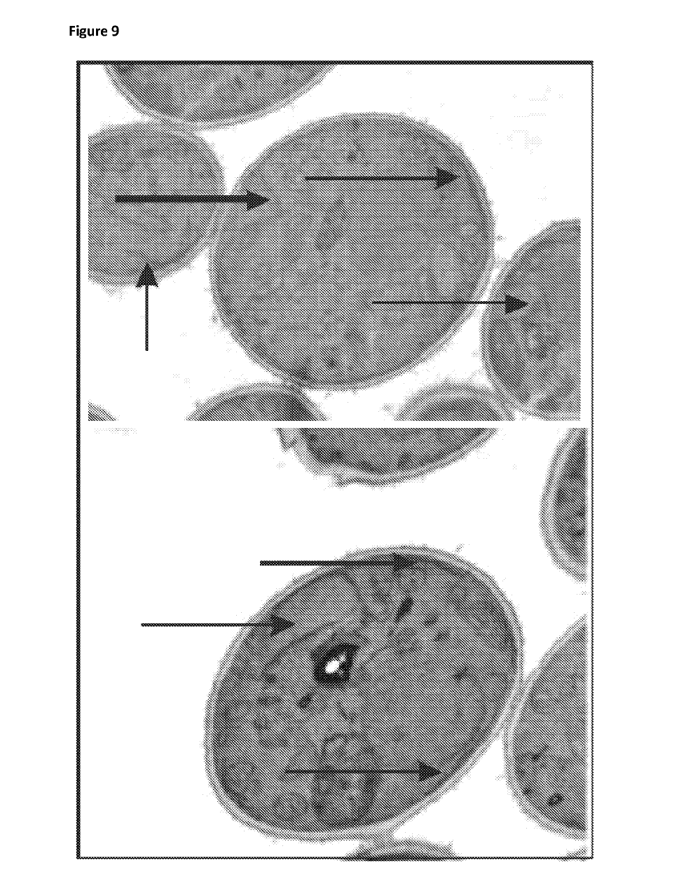

Membrane expansion of the engineered dgk1p overexpressing strain was evaluated by electron microscopy after 24 h of induction of the dgk1p. It can be seen that the membrane phenotype is similar to the PAH1 knock-out (FIG. 9), as was previously reported for S. cerevisiae (Han et al., 2008a,b).

### Example 3

### Disruption of the PAH1 Gene of P. pastoris

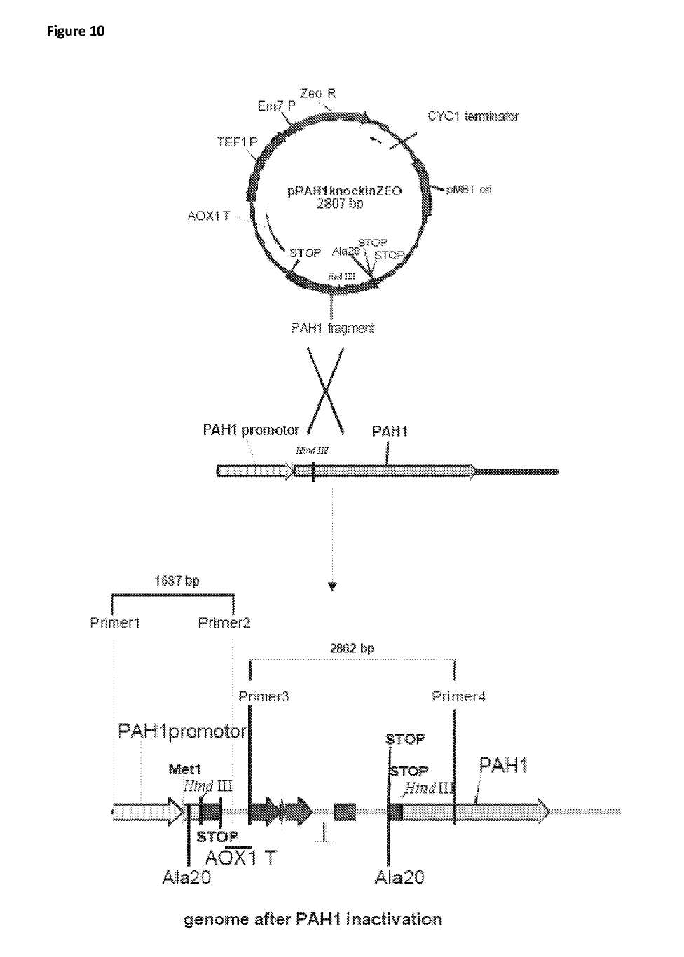

A knock out strain of the PAH1 gene in P. pastoris was created. As double homologous recombination at this locus seems to occur very rarely, it was decided to create a disruption cassette to disrupt the gene rather than to knock out the gene. The PAH1 gene of a P. pastoris expressing the Adenosine A2A receptor was disrupted after transformation of the plasmid pPAH1knockinZEO. Positive clones were identified by PCR. The cloning strategy is shown in FIG. 10. Briefly, after digestion of the plasmid pPAH1knockinzeo with HindIII and transformation, the plasmid integrates in the PAH1 locus. Integration of the vector results in a short fragment not containing the catalytic active domain of the PAH1 gene and a fragment that cannot be translated because of the absence of a promoter and the presence of 2 in frame nonsense codons. Integration of the plasmid was confirmed via PCR. A first PCR (primer 1 and primer 2) results in a fragment of 1687 bp and a second PCR results (primer 3 and primer 4) in a fragment of 2862 bp. PCR fragments were isolated and sequenced to confirm correct integration.

### Growth Analysis of PAH1 Disruption Clone

Growth at 30° C. and 37° C. was evaluated. As expected, a slower growth was observed at 37° C. in agreement with the temperature sensitivity seen for the Δpah1 mutant of S. cerevisae. At 30° C. no growth defect is seen comparable with the observations of the dgk1p overexpression (Example 2) (FIG. 11).

### Evaluation of the Membrane Expansion in the PAH1 Disruption Clone

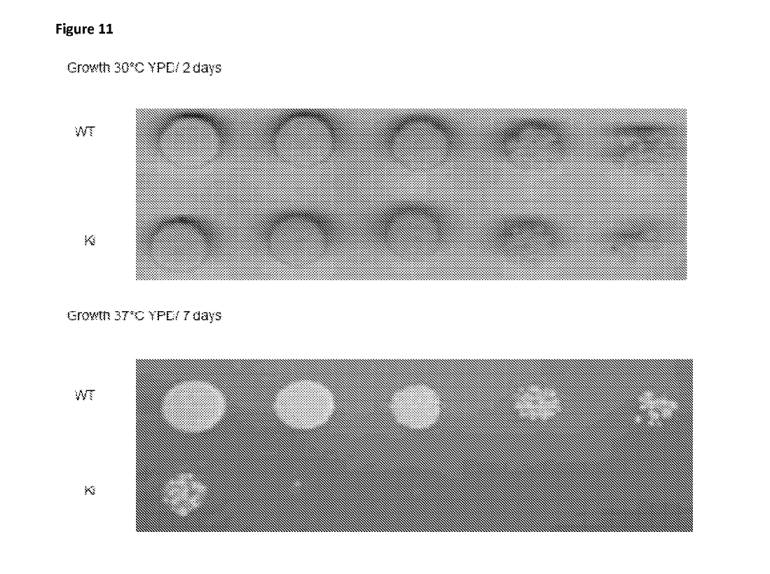

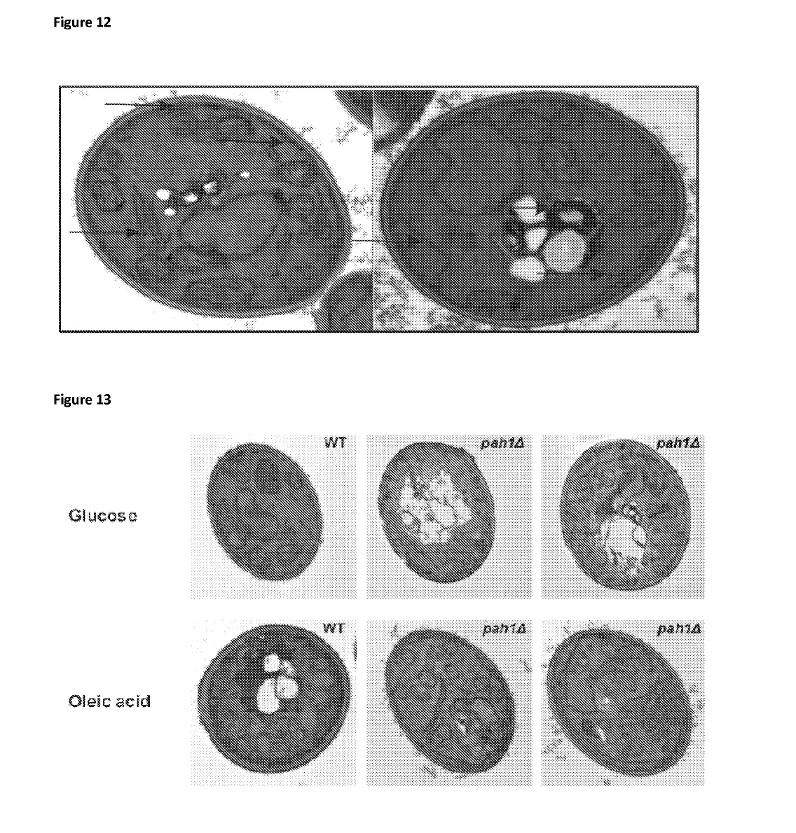

Membrane expansion was evaluated through EM (FIG. 12). Here also, extra membranes are observed. The membranes of these yeast cells appear often closely associated with phagosomes. These data suggest that the membranes might be eliminated over time. When P. pastoris cells are grown on oleic acid instead of glucose, the ER membrane increase is more pronounced, and not associated with vacuoles (FIG. 13). Thus, a strategy to increase the effect of the PAH1 KO on membrane protein expression might be to express the receptor from a fatty acid inducible promoter.

### Evaluation of Expression Levels of A2AR in the PAH1 Disruption Clone

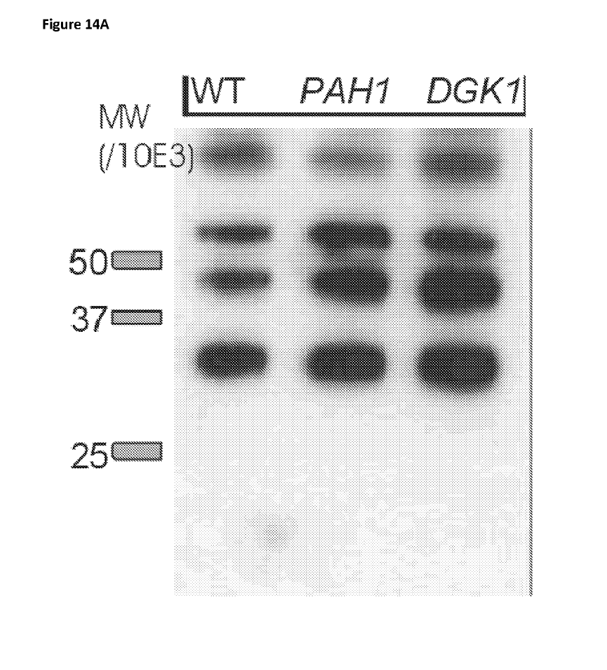

The expression levels of the receptor A2AR were evaluated after 24 hours of induction on methanol containing medium on Western Blot. The obtained expression levels are comparable with the levels obtained in the dgk1p overexpressing strain and slightly higher than the WT strain (FIG. 14A).

### Example 4

### Functionality of Expressed Proteins

To test whether the GPCR produced in the mutant Pichia pastoris strains is also functional, ligand binding assays were performed on the expressed adenosine 2A receptor. The results are shown in FIG. 14B.

The ligand binding of the A2AR expressed in the WT, PAH1 and DGK1 strain was evaluated by a radioligand binding saturation assay. The 3 strains show a comparable ligand binding and the produced GPCR thus is functional. Although it may seem surprising that the higher expression level is not correlated to an increase in active binding receptor, this may be explained by the already high expression levels of A2AR in the wild type Pichia strain: a protein that expresses lower quantities in the wild type strain will likely result in different activities.

### Example 5

### Evaluation of the Expression Level of the A2A Receptor in a PAH1 Knock Out Yarrowia lipolytica Strain

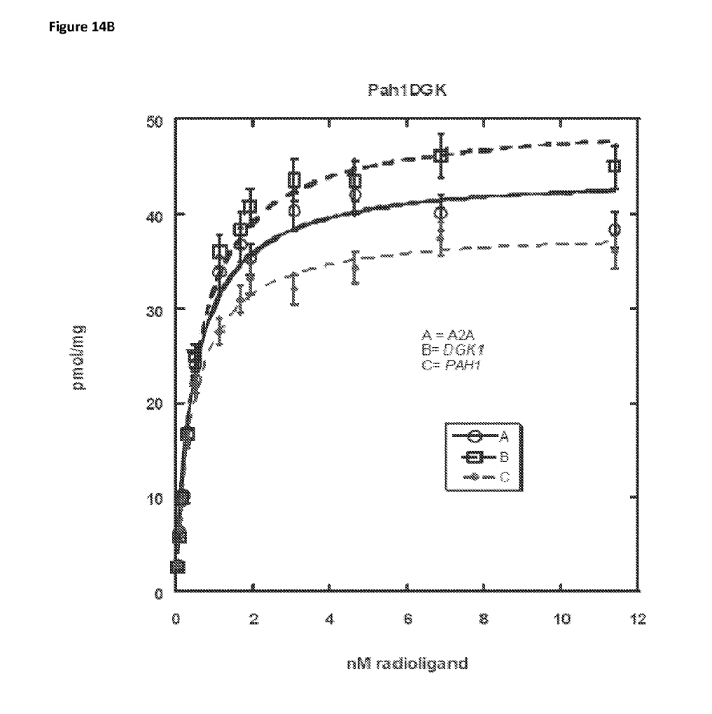

A PAH1 knock out strain was created in a PO1D(Leu-) strain overexpressing the Adenosine A2A receptor. The knock out strategy is described in the material and methods section. Expression levels of the receptor were analyzed on a Western Blot (shown in FIG. 15A) and ligand binding of the receptor was determined by radioligand binding assays (FIG. 15B). The PAH1 knock out strains shows higher expression levels of the receptor. These do not strictly correlate with the ligand binding data. As mentioned above, this may be due to the already high expression levels of A2AR and thus maximum functionality. Additionally, in this case oleic acid was used to induce the cells, which may have altered the lipid environment and rendered part of the produced receptors conformationally inactive. This problem is easy to overcome by choosing a different induction strategy that does not influence the membrane lipid environment.

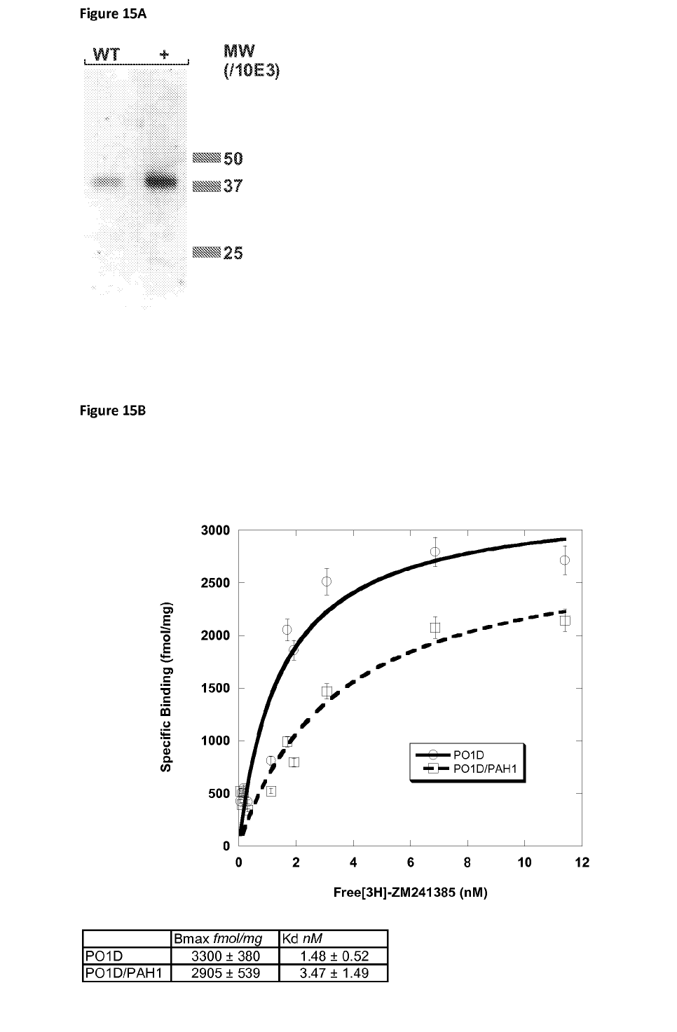

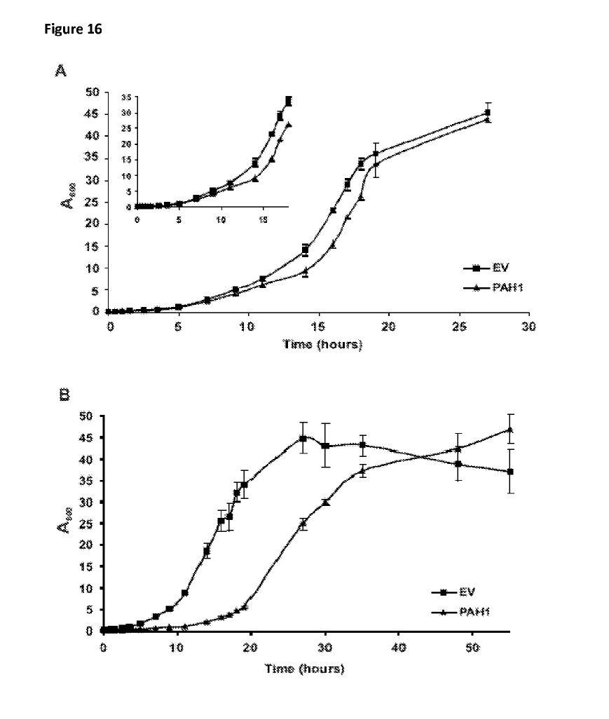

The experiment was repeated using cells grown on oleate and glucose as a carbon source. Because it is described that a PAH1 deletion strain of S. cerevisiae has a slower growth, we analyzed the growth of a Y. lipolytica ΔPAH1 strain. From FIG. 16 it can be seen that cells exhibited a different growth pattern dependent on the carbon source. Only a slight growth retardation is seen on glucose, especially in the early exponential phase. Cells grown on oleic acid show a remarkable longer lag phase but achieve the stationary phase at a same optical density as the wild type strain. Indeed it has been reported that S. cerevisae strains defective in neutral lipid synthesis (ΔARE1, ΔARE2, ΔDGA1, ΔLRO1) show an extended lag phase but can adapt to oleate growth. The authors speculate that this is due to the activation of gain-of-function suppressor genes (Connerth et al., 2010).

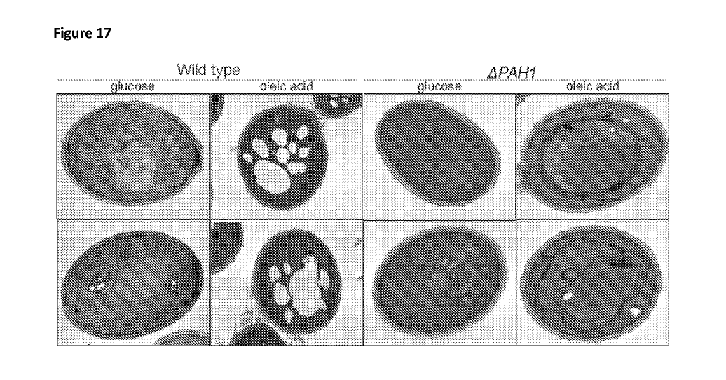

When achieving protein production, it was again observed that the deletion strain displays much higher protein production than the wild type (WT) strains and this as well on oleic acid as glucose. The increase on oleic acid is higher than on glucose (FIG. 18). This might be explained by the higher membrane expansion seen in these cells as can be seen from electron microscopy pictures taken at the end of protein induction (FIG. 17). One should also keep in mind that the initial expression levels for the strains expressing the A2A receptor from the hp4d promoter is higher than for the POX2 promoter.

Big lipid droplets are observed in the WT strain grown on oleic acid while the ΔPAH1 strains has none or very little and small lipid droplets, which is consistent with its defect in neutral lipid biosynthesis. The ER membrane proliferation in the strain grown on oleic acid medium is massive (FIG. 17).

### Example 6

### Production of VLPs, Budding from the Endoplasmic Reticulum, which Contain a Protein(s) of Interest

Here we describe the production of VLPs which bud from the internal membranes, more specifically from the ER. As the elevated PA content (resulting from PAP inhibition and/or increase in DGK activity) results in the increase of internal membrane, more VLPs will be able to bud from the ER of this engineered strain. One example is the expression of Hepatitis B surface antigen (HBsAg) from Hepatitis B virus (HBV). Particles containing HBsAg purified from plasma of asymptomatic HBV carriers, have already been used for vaccination since the early eighties. However, this type of vaccines is not frequently used for mass immunization due to safety concerns, restricted supply and costs.

It has already been shown by others (Gurramkonda et al., 2009; Hadiji-Abbes et al., 2009) that the overexpression of HBsAg alone is sufficient to produce ER-derived intracellular VLPs in Pichia pastoris. Upon co-expression of HBsAG from the Hepatitis B Virus (HBV) and a protein of interest in yeast, VLPs containing the protein of interest can be obtained.

### Cloning of HBsAg of the Hepatitis B Virus

The full-length sequence of HBsAg is obtained as a synthetic gene and amplified by PCR. The gene is cloned in a vector containing the zeocin resistance marker using the BstBI and NotI restriction sites. The vector is linearized in the AOX1 promoter with PmeI and transformed via electroporation to P. pastoris. Integration of the plasmid is confirmed through PCR on genomic DNA.

### Production and Purification of HBsAg-Based VLPs.

Clones are grown to saturation (±48 h) on BMGY at 30° C., collected by low-speed centrifugation and washed once with 1×PBS (Phosphate-Buffered Saline). They are then transferred to BMMY (1% methanol) for another 1-24 h at 30° C. Different lengths of induction times are possible, induction can be performed before or after spheroplast formation. Methanol is added every 12 h to sustain protein induction.

The yeast cells are ruptured upon heavy vortexing with glass beads for 5 times 3 minutes at 4° C. The debris is discarded from the lysate after centrifugation at 2,000 rpm for 20 minutes at 4° C. The supernatant is transferred to a clean microcentrifuge tube and BCA-assay is performed to estimate the total protein concentration.

### Evaluation of HBsAg-Based VLPs.

The supernatant is analyzed by SDS-PAGE and subsequent western blotting using rabbit anti-HBsAg antiserum and anti-rabbit IgG-HRP for visualization. Quantitation is performed by Coommassie brilliant blue staining and western blot on the supernatant, compared to serial dilutions of recombinant soluble HBsAg protein. An antibody against the protein of interest can be used to evaluate expression of this protein.

The production of VLPs is analyzed by electron microscopy on purified HBsAg as described previously by O'Keefe and Paiva, 1995; and Gurramkonda et al., 2009.

### Example 7

### Production of VLPs, Budding from the Plasma Membrane which Contain Adenosine A₂A Receptor

Here we describe the production of VLPs which bud from the plasma membrane. As the elevated PA content (resulting from PAP inhibition and/or increase in DGK activity) results in the increase of internal membrane, more membrane protein-containing VLPs will be formed. We describe the production of VLPs containing adenosine A₂A receptor as a protein of interest upon co-expression of the major structural protein Gag from the Human Immunodeficiency Virus (HIV) type-1 and the adenosine A₂A receptor in yeast.

It has been shown that the expression of Gag protein alone is sufficient to produce Gag VLPs that are morphologically identical to the immature form of HIV particles (Smith et al., 1993). The VLPs bud from the plasma membrane of eukaryotic cells when a large multimer is formed, resulting in a Gag-protein core enclosed by a plasma membrane-derived lipid bilayer (Tritel and Resh, 2000). This plasma membrane can be enriched for the adenosine A₂A receptor (or, if desired, other protein(s) of interest) by recombinant overexpression. Without organelle-specific targeting signals, a membrane protein (MP) or membrane-associated protein (MAP) is transported to the plasma membrane from which VLPs can bud. These VLPs can then be used for all kinds of applications: vaccines, structural biology (crystallography, NMR, . . . ), drug screening, ligand binding, panning of antibody/nanobody libraries . . . .

### Cloning of GAG of the Human Immunodeficiency Virus Type-1.

The full-length sequence of Gag from HIV-1 is obtained as a synthetic gene and amplified by PCR using a forward primer, 5′-TTCGAAATGGGTGCGAGAGCGTCAGT-3′ (SEQ ID NO: 16), and a reverse primer, 5′-GCGGCCGCTTATTGTGACGAGGGGTC-3′ (SEQ ID NO: 17). The gene is cloned in a vector containing the zeocin resistance marker using the BstBI and NotI restriction sites. The vector is linearized in the AOX1 promoter with the restriction enzyme PmeI and transformed via electroporation to a P. pastoris strain expressing the Adenosine A₂A receptor under control of the AOX1 promoter. Integration of the plasmid is confirmed through PCR on genomic DNA.

### Production and Purification of Gag-Based VLPs.

Positive clones are evaluated in first instance for the presence of VLPs. Clones are grown to saturation (±48 h) on BMGY at 30° C., collected by low-speed centrifugation and washed once with wash buffer (50 mM Tris, pH7.5, 5 mM MgCl2, 1M sorbitol). The cells are resuspended in wash buffer containing 30 mM DTT and incubated at 30° C. for 20 min with gentle agitation. The cells are then transferred to wash buffer containing 3 mM DTT and Zymolyase-100T (AMS Biotechnology, UK) at a final concentration of 0.4 mg/ml. The reaction is allowed to proceed at 30° C. for 20 min with gentle agitation. After being washed twice with 1M sorbitol, spheroplasts are cultured at 30° C. for another 1-24 h in BMMY (1% methanol). Different lengths of induction times are possible, induction can be performed before or after spheroplast formation. Methanol is added every 12 h to sustain protein induction.

The culture medium of the yeast spheroplasts, in which the VLPs are secreted, is clarified by low-speed centrifugation and then centrifuged through 30% (w/v) sucrose cushions at 4° C. at 26,000 rpm for 90 min. The VLP pellet is resuspended with 1×PBS (Phosphate-Buffered Saline) and centrifuged on 20-70% (w/v) sucrose gradient at 4° C. at 35,000 rpm overnight. Purified VLPs are obtained by fractionation of the gradient (Protocol after Sakuragi et al., 2002).

### Evaluation of Gag-Based VLPs.

The fractions are analyzed by SDS-PAGE and subsequent western blotting using anti-Gag mAb for Gag, anti-Rho1D4 mAb for the adenosine A2A receptor, and anti-mouse IgG-HRP for visualization. Quantitation is performed by Coommassie brilliant blue staining and western blot on the different purified VLP-fractions, compared to serial dilutions of recombinant soluble Gag protein and adenosine A₂A receptor.

### Material and Methods

### Strains Culture Conditions and Reagents:

Escherichia coli strains MC1061 were used for the amplification of recombinant plasmid DNA and grown in a thermal shaker at 37° C. in Luria-Broth (LB) medium supplemented with 50 μg/ml of kanamycin. Yarrowia lipolytica PO1D (leu2/Ura3) strain transformed with the plasmid 5HT1D(POX/URA) and JMP62-5HT1D. Plasmids bearing the 5HT1D receptor respectively with and without lip2 prepro secretion signal. Four clones expressing a different amount of expression cassettes of the 5HT1D receptor were evaluated. Pichia pastoris strain GS115 (his4) transformed with the plasmid pPIC92A2A. Po1dΔOCH1 (MatA, leu2-270, ura3-302, xpr2-322, Δoch1). Yeast were standard grown in YPD (1% yeast extract/2% Peptone/2% dextrose).

### Small Scale Protein Induction

Induction experiments were performed in a 125 ml baffled flask with 12.5 ml culture. For all experiments a preculture was grown and cells were inoculated at an OD 0.1. The A2A receptor expressed from the hp4 promoter was grown for 24 hours in YTG (1% yeast extract/2% Tryptone/2% dextrose). For induction on oleic acid, cells were grown for 24 hours in YTG, washed once with water and than resuspended in induction medium (50 mM Phosphate buffer pH 6.8/1% yeast extract/2% Tryptone/2% oleic acid). Cells were grown for 24 hours before harvesting.

### Construction of the Knock Out Plasmid and Strains:

A PAH1 knock out construct was made according to Fickers et al (Fickers et al., 2003). In short, a 549 bp promoter (primer sequence: pah1ylPfw07-010: 5′GCGGCCGCGAAGACGGTGAGTATGGCCATC 3′ (SEQ ID NO: 12) and pah1ylPrv07-006: 5′TAGGGATAACAGGGTAATCCATTGACACAGAACTCGACCT 3′ (SEQ ID NO: 18)) and a 551 bp terminator fragment (primer sequence: pah1ylTfw07-011: 5′ ATTACCCTGTTATCCCTACCGTACTGTACACCGTAGTTTG 3′ (SEQ ID NO: 19) and pah1ylTrv07-007: 5′ GCGGCCGCCCAAACCATGCATACAAATCAG 3′ (SEQ ID NO: 13)) were amplified from genomic DNA using PCR. DNA was amplified with Phusion polymerase (Finnzymes) The reverse primer of the promoter fragment and the forward primer of the terminator fragment contain the rare meganuclease I-SceI recognition site which can be used in a fusion PCR to obtain a PT fragment. (Promotor I-SceI and I-SceI terminator resulting fragments are then pooled and used for amplification of the Promotor-I-SceI-Terminator cassette). This fragment is then cloned as a blunt-ended fragment into the pCR-Blunt II-TOPO vector (Invitrogen) resulting in a pCR-Blunt-TOPO PT vector. For the construction of the final Promotor-selection marker-Terminator fragment, the plasmid was digested with the I-SceI meganuclease enzyme for rescue of the marker. In a next step the marker is cloned into the pCR-Blunt-TOPO PT vector after I-SceI digestion. Linearization by a NotI digest of the final plasmid, results in the final disruption cassette containing a promoter-leucine marker-terminator fragment. Disruption of the PAH1 gene occurs by double homogenous recombination.

Construction of the Pichia pastoris PAH1 Disruption Plasmid pPAH1knockinzeo:

A fragment of 151 AA (amino acids) from Ala20 to Thr151 was isolated via PCR with primers PAH1pp09-023 (5′-agatcttaataagccacactgagtggtgctattg-3′) (SEQ ID NO: 20) and PAH1pp08-02 (5′-GCGGCCGCTTACGTATTCTCAGGACTTGAGCTCAC-3′ (SEQ ID NO: 21)) introducing a BglII and NotI site respectively for cloning. The fragment was cloned in the pGlycoSwitchM8 plasmid (Vervecken et al applied and environmental microbiology 2004) by replacing the Δoch1 disruption sequence.

### Transformation:

Transformation of competent cells of Yarrowia lipolytica was performed as described in Boisramé et al., 1996.

Pichia pastoris cells were transformed according to the protocol from the Pichia Expression kit (Invitrogen Cat. No. K1710-01).

### Southern Blot:

Genomic DNA was prepared using the Epicenter Kit (Epicenter Biotechnologies, Madison, Wis.). Southern was performed according to the kit insert from Amersham Gene Images AlkPhos Direct labeling and Detection System. The probe was an I-SceI/AvaI fragment from the vector pah1PLTpah1inverse. 15 μg DNA was loaded on a 1% agarose gel and blotted overnight to a Hybond-N+ membrane (Amersham) in 10 SSC. After blotting, the membrane was cross-linked to the membrane with a UV-cross linker.

### Induction:

Yarrowia lipolytica: From a fresh plate a single colony was inoculated in 12.5 ml YTD (1% yeast extract/2% trypton/2% dextrose) in a 125 ml Erlenmeyer and grown for 24 hours at 28° C. and 250 rpm. After 24 hours, cells were washed once with Sodium-phosphate buffer pH 6.8 and resuspended in induction medium containing 4% oleic acid (Sodium phosphate buffer pH 6.8/1% yeast extract/2% Tryptone/5% oleic acid). Induction was performed for 24 hours at 28° C. and 250 rpm. At the end of the induction OD600 was measured. To avoid interference of not used oleic acid; the pellet was washed twice with UP water before OD measuring.

Pichia pastoris: Cells were inoculated in a total of 12.5 ml BMGY in a 125 ml shake flask and grown for 48 h. Cells were washed once with BMY, resuspended in BMMY, and induced for 48 h. Methanol (100%) was added every 8-12 h to a final concentration of 1% to maintain the induction. The adenosine A2A receptor was induced for 24 h before harvesting for analysis.

### Growth Curve

Cultures were grown overnight to saturation in YPD and diluted next day to an OD600 of 0.2. Incubation was continued and OD600 was measured at different time points. For the cultures grown in medium containing oleic acid cells were spun down for 10 min at 13,000 rpm and washed once with 0.1 N NaOH in order to avoid interference of oleic acid on the OD600 measurement. OD600 of different cultures in different media were measured in triplicate.

### Membrane Preparation:

Membranes were prepared from cells that have been induced for 24-27 h. Cells were pelleted by centrifugation at 1500×g and the pellet was resuspended in ice-cold breaking buffer (50 mM sodium phosphate buffer pH 7.4, complete protease inhibitor Roche/5% glycerol). Cells were then broken by vigorous vortexing with glass beads in a mixer mill for 10 times 1 min or 5 times 2 min at 4° C. Cells were separated from the membrane suspension by low speed centrifugation (2000 rpm, 5 min, 4° C. or 1000×g, 30 min, 4° C.). Membranes were pelleted at 100,000 g and 4° C. for 60 min and resuspended in resuspension buffer (50 mM sodium phosphate buffer pH 7.4/2% SDS supplemented with a complete protease inhibitor from Roche) and snap-frozen in liquid nitrogen until further testing. The protein concentration of the membrane preparation was determined using the BCA reagent (Pierce, Rockford, Ill.) with BSA as standard. Ten micrograms of total membrane protein was analyzed by western blot. The blot was blocked overnight in blocking buffer (0.05% Tween-20 and 3% casein in 1×PBS) and probed with a 1/500 diluted primary mouse anti-Rho1D4 antibody, followed by a 1/3000 diluted secondary anti-mouse IgG peroxidase from sheep (Sigma Cat. n^{o}NA931V). Protein bands were visualized with Renaissance western blot chemiluminescence reagent plus (PerkinElmer).

### Protein Analysis:

For each strain an equal amount of protein was dissolved in laemli buffer and heated for 10 min at 50° C. Samples were then loaded on a 12% SDS-PAGE. After electrophoreses samples were transferred to a nylon membrane (PerkinElmer). The blot was blocked overnight in blocking buffer (Tween-20 0.05%/3% casein/1×PBS) and probed with a 1/100 diluted primary mouse anti-Rho1D4 antibody followed by a 1/3000 diluted secondary anti-mouse IgG peroxidase from sheep (Sigma Cat. n^{o} NA931V). Protein bands were visualized with Renaissance Western blot chemiluminescence reagent plus (Perkin Elmer).

### Binding Assays:

Radioligand binding assays were performed according to Weiss et al (Eur J Biochem 2002, 269(1):82-92). Briefly, 5 μg of total membrane protein was incubated with different concentrations (0.06-14 nM) of the A2AR antagonist ³[H]ZM241385 in 500 μl binding buffer (20 mM HEPES pH 7.4, 100 mM NaCl). Adenosine deaminase (0.1 U) was added to degrade the adenosine released from the membranes and the membranes were incubated at 22° C. for 1 h. Non-specific binding was determined in the presence of 10 mM theophylline. After incubation, bound and free ligand were separated on Whatmann GF/B filters pretreated with 0.1% polyethylenimine using a Brandel cell harvester. The filters were washed three times with binding buffer and the amount of bound radioligand was measured on a liquid scintillation counter. The Kd and Bmax are determined by curve fitting using KaleidaGraph software (Synergy Software).

### Ligand Binding Studies A2A Receptor

The procedures for studying binding at recombinant A2A receptors have been described (Weiss et el., 2002, see above). Briefly, 10 μg of total membrane protein was incubated with different concentrations (0.01-8 n M) of the A2AR antagonist 3[H]ZM241385 in 500 μl binding buffer (20 mM HEPES pH 7.4, 100 mM NaCl). Adenosine deaminase (0.1 U) was added to degrade the adenosine released from the membranes and the membranes were incubated at 22° C. for 1 h. Non-specific binding was determined in the presence of 10 mM theophylline. Measurements were performed in duplicate. After incubation, bound and free ligand were separated on Whatmann GF/B filters pretreated with 0.1% polyethylenimine using a Brandel cell harvester. The filters were washed three times with binding buffer and the amount of bound radioligand was measured on a liquid scintillation counter. The Kd and Bmax are determined by curve fitting using KaleidaGraph software (Synergy Software).

### Electron Microscopy:

Samples were prepared for EM according to Baharaeen et al (Mycopathologia, 1982, 77(1): 19-22). For experiments wherein A2A receptor is expressed, EM pictures were taken at the end of induction of the A2A receptor. Briefly, electron microscopy was performed on an overnight grown yeast culture in YPD. Yeast cells were fixed for 2 hours on ice in 1.5% paraformaldehyde and 3% glutaraldehyde in 0.05 M sodiumCacodylate buffer, pH 7.2. After washing 3 times for 20 min in buffer, cells were treated with a 6% aqueous solution of potassium permanganate for 1 hour at room temperature. After washing 3 times for 20 min in buffer, cells were dehydrated through a graded ethanol series, including a bulk staining with 2% uranyl acetate at the 50% ethanol step, followed by embedding in Spurr's resin. Ultrathin sections of a gold interference colour were cut using an ultra microtome (ultracut E/Reichert-Jung), followed by a post-staining with uranyl acetate and lead citrate in a Leica ultrastainer and collected on formvar-coated copper slot grids. They were viewed with a transmission electron microscope 1010 (JEOL, Tokyo, Japan).

### REFERENCES

André N, Cherouati N, Prual C, Steffan T, Zeder-Lutz G, Magnin T, Pattus F, Michel H, Wagner R, Reinhart C. Enhancing functional production of G protein-coupled receptors in Pichia pastoris to levels required for structural studies via a single expression screen. Protein Sci. 2006; 15(5):1115-26.
Balliet J W, Bates P. Efficient infection mediated by viral receptors incorporated into retroviral particles. J Virol. 1998; 72(1):671-6.
Boisramé A, Beckerich J M, Gaillardin C. Sls1p, an endoplasmic reticulum component, is involved in the protein translocation process in the yeast Yarrowia lipolytica. J Biol Chem. 1996; 271(20):11668-75.
Bräutigam S, Snezhkov E, Bishop D H. Formation of poliovirus-like particles by recombinant baculoviruses expressing the individual VP0, VP3, and VP1 proteins by comparison to particles derived from the expressed poliovirus polyprotein. Virology. 1993; 192(2):512-24.
Carman G M, Han G S. Roles of phosphatidate phosphatase enzymes in lipid metabolism. Trends Biochem Sci. 2006; 31(12):694-9.
Carman G M, Han G S. Phosphatidic acid phosphatase, a key enzyme in the regulation of lipid synthesis. J Biol Chem. 2009; 284(5):2593-7.
Cherezov V, Rosenbaum D M, Hanson M A, Rasmussen S G, Thian F S, Kobilka T S, Choi H J, Kuhn P, Weis W I, Kobilka B K, Stevens R C. High-resolution crystal structure of an engineered human beta2-adrenergic G protein-coupled receptor. Science 2007; 318:1258-1265.
Chien E Y, Liu W, Zhao Q, Katritch V, Han G W, Hanson M A, Shi L, Newman A H, Javitch J A, Cherezov V, Stevens R C. Structure of the human dopamine D3 receptor in complex with a D2/D3 selective antagonist. Science 2010; 330(6007):1091-5.
Connerth M, Czabany T, Wagner A, Zellnig G, Leitner E, Steyrer E, Daum G. Oleate inhibits steryl ester synthesis and causes liposensitivity in yeast. J Biol Chem. 2010; 285(35):26832-41.
De Pourcq K, De Schutter K, Callewaert N. Engineering of glycosylation in yeast and other fungi: current state and perspectives. Appl Microbiol Biotechnol. 2010; 87(5):1617-31.
Delchambre M, Gheysen D, Thines D. et al. The GAG precursor of simian immunodeficiency virus assembles into virus-like particles. EMBO J 1989; 8:2653⁻⁶⁰.
Endres M J, Jaffer S, Haggarty B, Turner J D, Doranz B J, O'Brien P J, Kolson D L, Hoxie J A. Targeting of HIV- and SIV-infected cells by CD4-chemokine receptor pseudotypes. Science. 1997; 278(5342):1462-4.
Falcón V, García C, de la Rosa M C, Menéndez I, Seoane J, Grillo J M. Ultrastructural and immunocytochemical evidences of core-particle formation in the methylotrophic Pichia pastoris yeast when expressing HCV structural proteins (core-E1). Tissue Cell. 1999; 31(2):117-25.
Fickers P, Le Dall M T, Gaillardin C, Thonart P, Nicaud J M: New disruption cassettes for rapid gene disruption and marker rescue in the yeast Yarrowia lipolytica. J Microbiol Methods 2003, 55(3):727-737.
French T J, Marshall J J, Roy P. Assembly of double-shelled, virus-like particles of bluetongue virus by the simultaneous expression of four structural proteins. J Virol 1990; 64:5695-700.
Gheysen D, Jacobs E, de Foresta F et al. Assembly and release of HIV-1 precursor Pr55gag virus-like particles from recombinant baculovirus-infected insect cells. Cell 1989; 59:103-12.
Grimsey N, Han G S, O'Hara L, Rochford J J, Carman G M, Siniossoglou S. Temporal and spatial regulation of the phosphatidate phosphatases lipin 1 and 2. J Biol Chem. 2008; 283(43):29166-74.
Grisshammer R, Duckworth R, Henderson R. Expression of a rat neurotensin receptor in Escherichia coli. Biochem J. 1993; 295 (Pt 2):571-6.
Guerfal M, Ryckaert S, Jacobs P P, Ameloot P, Van Craenenbroeck K, Derycke R, Callewaert N: The HAC1 gene from Pichia pastoris: characterization and effect of its overexpression on the production of secreted, surface displayed and membrane proteins. Microb Cell Fact. 2010, 9:49.
Gurramkonda C, Adnan A, Gabel T, Lünsdorf H, Ross A, Nemani S K, Swaminathan S, Khanna N, Rinas U. Simple high-cell density fed-batch technique for high-level recombinant protein production with Pichia pastoris: Application to intracellular production of Hepatitis B surface antigen. Microb Cell Fact. 2009; 8:13.
Hadiji-Abbes N, Borchani-Chabchoub I, Triki H, Ellouz R, Gargouri A, Mokdad-Gargouri R. Expression of HBsAg and preS2-S protein in different yeast based system: a comparative analysis. Protein Expr Purif. 2009; 66(2):131-7.
Han G S, Wu W I, Carman G M. The Saccharomyces cerevisiae Lipin homolog is a Mg2+-dependent phosphatidate phosphatase enzyme. J Biol Chem. 2006; 281(14):9210-8.
Han G S, Siniossoglou S, Carman G M. The cellular functions of the yeast lipin homolog PAH1p are dependent on its phosphatidate phosphatase activity. J Biol Chem. 2007; 282(51):37026-35.
Han G S, O'Hara L, Carman G M, Siniossoglou S. An unconventional diacylglycerol kinase that regulates phospholipid synthesis and nuclear membrane growth. J Biol Chem. 2008a; 283(29):20433-42.
Han G S, O'Hara L, Siniossoglou S, Carman G M. Characterization of the yeast DGK1-encoded CTP-dependent diacylglycerol kinase. J Biol Chem. 2008b; 283(29):20443-53.
Hellwig S, Drossard J, Twyman R M, Fischer R. Plant cell cultures for the production of recombinant proteins. Nat Biotechnol. 2004; 22(11):1415-22.
Huang T-K, McDonald K A. Bioreactor engineering for recombinant protein production in plant cell suspension cultures. Biochemical Engineering Journal 2009; 45(3):168-184.
Insel P A, Tang C M, Hahntow I, Michel M C. Impact of GPCRs in clinical medicine: genetic variants and drug targets. Biochim Biophys Acta. 2007; 1768(4): 994-1005.
Jaakola V P, Griffith M T, Hanson M A, Cherezov V, Chien E Y, Lane J R, Ijzerman A P, Stevens R C. The 2.6 angstrom crystal structure of a human A2A adenosine receptor bound to an antagonist. Science 2008; 322:1211-1217.
Jacobs P P, Callewaert N. N-glycosylation engineering of biopharmaceutical expression systems. Curr Mol Med. 2009; 9(7):774-800.
Jose A M, Koelle M R. Domains, amino acid residues, and new isoforms of Caenorhabditis elegans diacylglycerol kinase 1 (DGK-1) important for terminating diacylglycerol signaling in vivo. J Biol Chem. 2005; 280(4):2730-6.
Katagiri T, Mizoguchi T, Shinozaki K. Molecular cloning of a cDNA encoding diacylglycerol kinase (DGK) in Arabidopsis thaliana. Plant Mol Biol. 1996; 30(3):647-53.
Kibenge F S, Qian B, Nagy E, Cleghorn J R, Wadowska D. Formation of virus-like particles when the polyprotein gene (segment A) of infectious bursal disease virus is expressed in insect cells. Can J Vet Res. 1999; 63(1):49-55.
Liu W, Jiang H, Zhou J, Yang X, Tang Y, Fang D, Jiang L. Recombinant dengue virus-like particles from Pichia pastoris: efficient production and immunological properties. Virus Genes. 2010; 40(1):53-9.
Lundstrom K, Wagner R, Reinhart C, Desmyter A, Cherouati N, Magnin T, Zeder-Lutz G, Courtot M, Prual C, André N, Hassaine G, Michel H, Cambillau C, Pattus F. Structural genomics on membrane proteins: comparison of more than 100 GPCRs in 3 expression systems. J Struct Funct Genomics. 2006; 7(2):77-91.
Madzak C, Gaillardin C, Beckerich J M. Heterologous protein expression and secretion in the non-conventional yeast Yarrowia lipolytica: a review. J Biotechnol 2004, 109(1-2):63-81.
Miyanohara A, Imamura T, Araki M. et al. Expression of hepatitis B virus core antigen gene in Saccharomyces cerevisiae: synthesis of two polypeptides translated from different initiation codons. J Virol 1986; 59:176-80.
Noad R, Roy P. Virus-like particles as immunogens. Trends Microbiol. 2003; 11(9):438-44.
O'Keefe D O, Paiva A M. Assay for recombinant hepatitis B surface antigen using reversed-phase high-performance liquid chromatography. Anal Biochem. 1995; 230(1):48-54.
Palczewski K, Kumasaka T, Hori T, Behnke C A, Motoshima H, Fox B A, Le Trong I, Teller D C, Okada T, Stenkamp R E, Yamamoto M, Miyano M. Crystal structure of rhodopsin: A G protein-coupled receptor. Science. 2000; 289(5480):739-45.
Penel S, Arigon A M, Dufayard J F, Sertier A S, Dau bin V, Duret L, Gouy M, Perrière G. Databases of homologous gene families for comparative genomics. BMC Bioinformatics. 2009; 10 Suppl 6:S3.
Péterfy M, Phan J, Xu P, Reue K. Lipodystrophy in the fld mouse results from mutation of a new gene encoding a nuclear protein, lipin. Nat Genet. 2001; 27(1):121-4.
Rasmussen S G, Choi H J, Rosenbaum D M, Kobilka T S, Thian F S, Edwards P C, Burghammer M, Ratnala V R, Sanishvili R, Fischetti R F, Schertler G F, Weis W I, Kobilka B K. Crystal structure of the human beta2 adrenergic G-protein-coupled receptor. Nature 2007; 450:383-387.
Reeves P J, Thurmond R L, Khorana H G. Structure and function in rhodopsin: high level expression of a synthetic bovine opsin gene and its mutants in stable mammalian cell lines. Proc Natl Acad Sci USA. 1996; 93(21):11487-92.
Reeves P J, Callewaert N, Contreras R, Khorana H G. Structure and function in rhodopsin: high-level expression of rhodopsin with restricted and homogeneous N-glycosylation by a tetracycline-inducible N-acetylglucosaminyltransferase I-negative HEK293S stable mammalian cell line. Proc Natl Acad Sci USA. 2002; 99(21):13419-24.
Roy P, Noad R. Virus-like particles as a vaccine delivery system: myths and facts. Hum Vaccin. 2008; 4(1):5-12.
Sakane F, Imai S, Kai M, Yasuda S, Kanoh H. Diacylglycerol kinases: why so many of them? Biochim Biophys Acta. 2007; 1771(7):793-806.
Sakuragi S, Goto T, Sano K, Morikawa Y. HIV type 1 Gag virus-like particle budding from spheroplasts of Saccharomyces cerevisiae. Proc Natl Acad Sci USA. 2002; 99(12):7956-61.
Salom D, Le Trong I, Pohl E, Ballesteros J A, Stenkamp R E, Palczewski K, Lodowski D T. Improvements in G protein-coupled receptor purification yield light stable rhodopsin crystals. J Struct Biol. 2006; 156(3):497-504.
Santi L, Huang Z, Mason H. Virus-like particles production in green plants. Methods. 2006; 40(1):66-76.
Santos-Rosa H, Leung J, Grimsey N, Peak-Chew S, Siniossoglou S. The yeast lipin Smp2 couples phospholipid biosynthesis to nuclear membrane growth. EMBO J. 2005; 24(11):1931-41.
Siniossoglou S, Santos-Rosa H, Rappsilber J, Mann M, Hurt E. A novel complex of membrane proteins required for formation of a spherical nucleus. EMBO J. 1998; 17(22):6449-64.
Smith A J, Srinivasakumar N, Hammarskjold M L, Rekosh D. Requirements for incorporation of Pr160gag-pol from human immunodeficiency virus type 1 into virus-like particles. J Virol. 1993; 67(4):2266-75.
Sugrue R J, Fu J, Howe J, Chan Y C. Expression of the dengue virus structural proteins in Pichia pastoris leads to the generation of virus-like particles. J Gen Virol. 1997; 78 (Pt 8):1861-6.
Tritel M, Resh M D. Kinetic analysis of human immunodeficiency virus type 1 assembly reveals the presence of sequential intermediates. J Virol. 2000; 74(13):5845-55.
Vaultier M N, Cantrel C, Guerbette F, Boutté Y, Vergnolle C, Ciçek D, Bolte S, Zachowski A, Ruelland E. The hydrophobic segment of Arabidopsis thaliana cluster I diacylglycerol kinases is sufficient to target the proteins to cell membranes. FEBS Lett. 2008; 582(12):1743-8.
Warne T, Serrano-Vega M J, Baker J G, Moukhametzianov R, Edwards P C, Henderson R, Leslie A G, Tate C G, Schertler G F. Structure of a beta1-adrenergic G-protein-coupled receptor. Nature 2008; 454:486-491.
Wildt S, Gerngross T U. The humanization of N-glycosylation pathways in yeast. Nat Rev Microbiol. 2005; 3(2):119-28.
Willis S, Davidoff C, Schilling J, Wanless A, Doranz B J, Rucker J. Virus-like particles as quantitative probes of membrane protein interactions. Biochemistry. 2008; 47(27):6988-90.
Wu B, Chien E Y, Mol C D, Fenalti G, Liu W, Katritch V, Abagyan R, Brooun A, Wells P, Bi F C, Hamel D J, Kuhn P, Handel T M, Cherezov V, Stevens R C. Structures of the CXCR4 chemokine GPCR with small-molecule and cyclic peptide antagonists. Science. 2010; 330(6007):1066-71.

1. A method of enhancing production of a protein in a eukaryotic cell, the method comprising the steps of:
providing a eukaryotic cell deficient in expression and/or activity of an endogenous phosphatidic acid phosphatase, and/or overexpressing a diacylglycerol kinase, the eukaryotic cell comprising a nucleotide encoding the protein, under conditions suitable for expressing the protein so as to express the protein.

2. The method according to claim 1, wherein the nucleotide is an exogenous nucleotide or an endogenous nucleotide under control of an exogenous promoter.

3. The method according to claim 1, wherein the eukaryotic cell is deficient in expression and/or activity of endogenous phosphatidic acid phosphatase, which is PAH1 or a homolog thereof.

4. The method according to claim 1, wherein the eukaryotic cell is deficient in expression and/or activity of endogenous phosphatidic acid phosphatase through disruption of the endogenous phosphatidic acid phosphatase gene at the nucleic acid level.

5. The method according to claim 1, wherein the eukaryotic cell is deficient in expression and/or activity of endogenous phosphatidic acid phosphatase through an inhibitory RNA directed to the endogenous phosphatidic acid phosphatase gene transcript.

6. The method according to claim 1, wherein the eukaryotic cell overexpresses a diacylglycerol kinase, and the diacylglycerol kinase is DGK1 or a homolog thereof.

7. The method according to claim 1, wherein endogenous diacylglycerol kinase is overexpressed.

8. The method according to claim 1, wherein an exogenous diacylglycerol kinase is overexpressed.

9. The method according to claim 1, wherein the overexpression of diacylglycerol kinase is inducible.

10. The method according to claim 1, wherein the protein is a membrane-bound protein.

11. The method of claim 1, wherein the protein is a receptor.

12. The method of claim 1, wherein the eukaryotic cell is a yeast cell and the protein is to be expressed and isolated from the cell.

13. The method according to claim 12, wherein the yeast cell is a glyco-engineered yeast cell.

14. The method of claim 1, wherein the eukaryotic cell is a mammalian cell.

15. The method according to claim 1, further comprising the step of isolating the expressed protein.

16. A method of enhancing production of a virus-like particle in a eukaryotic cell, the method comprising the steps of:
providing a eukaryotic cell deficient in expression and/or activity of an endogenous phosphatidic acid phosphatase, and/or overexpressing a diacylglycerol kinase, the cell comprising one or more nucleotides encoding the one or more proteins making up the virus-like particle, in conditions suitable for expressing the one or more proteins.

17. The method according to claim 16, wherein the virus-like particle further encompasses lipids.

18. The method according to claim 16, wherein the virus-like particles are used for production of vaccines or for production of membrane proteins.

19. The method according to claim 16, wherein the virus-like particles are based on a hepatitis virus, HCV virus, encephalitis virus, Japanese encephalitis virus, or Dengue virus.

20. A eukaryotic cell deficient in expression and/or activity of an endogenous phosphatidic acid phosphatase, and/or overexpressing a diacylglycerol kinase, wherein if the eukaryotic cell is a Saccharomyces cell, it comprises an exogenous nucleotide, or an endogenous nucleotide under control of an exogenous promoter, encoding a protein to be expressed and isolated from the cell.

21. The eukaryotic cell according to claim 20, wherein the cell is a yeast cell.

22. The eukaryotic cell according to claim 20, wherein the endogenous phosphatidic acid phosphatase is PAH1 or a homolog thereof and/or wherein the diacylglycerol kinase is DGK1 or a homolog thereof.

23. A cell culture of cells according to claim 20.

24. The method according to claim 11, wherein the protein is a GPCR.

25. The method according to claim 12, wherein the yeast cell is a Yarrowia or Pichia cell.

26. The method according to claim 14, wherein the eukaryotic cell is a Hek293S cell.

27. The method of claim 17, wherein the virus-like particle is a lipoparticle.

28. The eukaryotic cell of claim 21, wherein the yeast cell is a Yarrowia or Pichia cell.


SEQUENCE LISTING (21 sequences)

SEQ ID NO: 1 - DNA, 3772 bp, Yarrowia lipolytica
aggctgtatttctgtgtgctagtcggcacttttcatccactccccacgtgatcacatcgattggaacagcaaacccctccttgtacttgctgcatatctgtatgtataccgaactctgtgaaggtgcccaacagatcatcaccacacaaccacacgatgcagtacgttgggcgagccattggaaccgtttccaagacctggaactccatcaaccctgcaacactcagtggtgcaattgacatcattgtggtcgagcgggaagacggtgagtatggccatcaagaggctgttggagaagtggagctgatgcagtgaccgacggtgacttacaggatgcaagttgagagggtgatagagaagtggactttgagaacagagagtgggtgcaagtggccttacagttgttacttctaactggcagttacaccactcttgtaactatcatggtccgtatgatgtgaccacatgttgtacaccatgactgtaatcgtgcccaggtggcaacagtatgattcacagttgttatgtcatatggacaaagttgtaagaggccaatacaactggagcaaccagcttgtttcgggtttcctgtcaactacttcccactcactcgaaacactcaacacatgaacacaacgtggtggcgtaacacctcatccccataaccaccatatccacacactaacccaggtgaactaaactgctctcctttccatgtgcggtttggaatcttttccctgctgcgaccgtcccagaaaaaggtcgagttctgtgtcaatggcaccaagaccgagctgcccatgaaggtgggcgatggaggagaagtcttctttgtgttcgagaccgacgcagacgtgcccgaagagctcctgacatcccccgtcatttctccctcttcgtcgccatcctggggccaggaggaaggcggggatggtgagccggactaccttgctctgaacgactctaaacagggtggcgacagcaagcacggcagatcgccctcggagggcccaccattcagatcaccttcggcggatcacttacatgagatgggcagcttcgatgatgagaatgaccctgaggtgaacagaagacaacgtgcgagcacggcagctccagagcctgttcctggttcgttgaaacacccagccactatctcggaaggcatctcttcggcctcgttttccaacagcgatactgatcgaacagacacttctggacccacagagacagaacccacagagctcacagagcctacagagcccacagagcccacagagcctctggatcttgagcagagtctccaccgggctgccacttctcccgccccttcgtccgaggagatttgggagaaggcccgtgcactgtccaagaaactcacatcagaaaacattcagagtaaaatctccgacaacggagacattattctggatatgactggttacaagtacgaccacgaggacgtgagtcgatcagaggagctggtcaagaaaatcctcgctgaggaactgggagaagacagagacctgtcccacatcctggttgaagacgaggagggtaaccttgtgattcagagcgctggagacagccaccatcacgagcatatgagctcgcccgagtctctggcccactcccctcagcccctcccttcttctaaccttccgtctcaggcctcggacaacaagcactacgccaagaccatccgtttgacgtctgaccagctcaagtctctggatctcaagcccggcaagaacgaggtcacctttgctgtcaataacggcaagacgtcgtgttcggcccagctgttctactggaagtacgacattcctgttgtcatttccgacattgatggcacgatcaccaagtccgatgctctgggccatctgctcaccatgatgggccgagactggacccacaccggcgtggccaagctcttttccgatatcagagccaacgggtataatatcatgtatctgacagcacgatcagtgggacaggcagatgcaaccagggcatatctaggcggtgttgaccagtttggcttcaagctgcctccaggacccgtcatcttgtcgcctgatagaaccctggcggctctcaagagagaggtgattcttaagaaacctgaggtattcaagatggcgtgtctgcgggacattaagtcgctgtttggcgagaccgaagacgccaccaatccattctacgctggatttggcaaccgaatcaccgacgcgttgtcgtatagatctgtcggtgtgccgtcgtctagaatcttcacaatcaactcgaacgccgaggtccatatggagctgcttgaactggctggctacaagtcctcgtatgtccacattgccgatcttgtcgaccactttttccctccggaaagcgagttcacgaccattcaggaggaaaaatacacggacgtcaactactggcgagatcccattattgacctgtctgatctgaccgacgacgagctgactgacgatgatgagctctccaagtcgcccaagtcgcccagatctcctagaagcccgcgggccggttcggcaggctccagcgcggctccctcaggctcgggcgccgaccctgccggaccctccgagccgaaggactccgcgaacccgtcgaagttcagctataagaaggctcctacgaactctcgattccagcccgtttcgtacgatcttgatcttgacgacggatacgagtacgacgatgacgatgactatgatgacgatgaggagtttgtggacgctgagagcgacgcgctggaggaggatgacgacgatgatgatgacgtcgacctagacaacgactctgaccactcccctgtcaagccgccctcgcagatgcagcgagtcatcaacaagactattgaggacaacaagggcctgcacatggatgaggatgacgttcaaaaagccatgaaggccctgaagatggaacgagcaagcatcaatcctgagtaaataggaaatcaagcataaatatatccgtactgtacaccgtagtttgtgacgtcgaaggggagggggacaagagagtgaaagtgtagctacttgtagtatgtataagttgtgcgtacctgaatgtcacgggacctgcgatacgaatgctggtgagacccaaccatggttcccgctagaaagccaagatattatctggatctacagctgagtttgacgttatttttttttccctcctgaaaggctttgttaggtttgcatcgttcatgtatttggtgtacacctttttcgggttccgatggtgcttccataggtggaggagcaggtcagagacacattgtttgtggatagcggattgttatgccccgagtggagcatcattttaatcgcaggagtttattatatactgtgtatactgtgcactaatttttcccctgtctcctctacactcccttacgtcattaaatggccgctactgtattcacactcttccgacccggatcagataaatttttcaagaacttttttatctgatttgtatgcatggtttgggggcaagtaagccgtacataggaggcctggaagtaggggcaaaagtggagagtccctgtaaccactggtttgtgcaattttgggggtcgcaaatacattgccacaacgtgggcgccaggagtttacagacgagtctatatacgggcggtcttagatgaattttactactgcacgtgtgagcaatttgagggcctgtgttgacgggtgcgagtttaacgtgcagggttcagtggggtgatttgagccgacaa

SEQ ID NO: 2 - protein, 723 aa, Yarrowia lipolytica
MKVGDGGEVFFVFETDADVPEELLTSPVISPSSSPSWGQEEGGDGEPDYLALNDSKQGGDSKHGRSPSEGPPFRSPSADHLHEMGSFDDENDPEVNRRQRASTAAPEPVPGSLKHPATISEGISSASFSNSDTDRTDTSGPTETEPTELTEPTEPTEPTEPLDLEQSLHRAATSPAPSSEEIWEKARALSKKLTSENIQSKISDNGDIILDMTGYKYDHEDVSRSEELVKKILAEELGEDRDLSHILVEDEEGNLVIQSAGDSHHHEHMSSPESLAHSPQPLPSSNLPSQASDNKHYAKTIRLTSDQLKSLDLKPGKNEVTFAVNNGKTSCSAQLFYWKYDIPVVISDIDGTITKSDALGHLLTMMGRDWTHTGVAKLFSDIRANGYNIMYLTARSVGQADATRAYLGGVDQFGFKLPPGPVILSPDRTLAALKREVILKKPEVFKMACLRDIKSLFGETEDATNPFYAGFGNRITDALSYRSVGVPSSRIFTINSNAEVHMELLELAGYKSSYVHIADLVDHFFPPESEFTTIQEEKYTDVNYWRDPIIDLSDLTDDELTDDDELSKSPKSPRSPRSPRAGSAGSSAAPSGSGADPAGPSEPKDSANPSKFSYKKAPTNSRFQPVSYDLDLDDGYEYDDDDDYDDDEEFVDAESDALEEDDDDDDDVDLDNDSDHSPVKPPSQMQRVINKTIEDNKGLHMDEDDVQKAMKALKMERASINPE

SEQ ID NO: 3 - DNA, 4325 bp, Pichia pastoris
tcatagtgcatcctcttgcactattgacaataatcgacgagttccagagacgaggtcgcaacaacgattccataatattcggtgggttacttggtaaacatgatgaatccaccaaccaaatatctgttgttaacagctttgtgataccattgatcgataatcagtttttgaataaagagtacttgcaggacatgctactcaaattttctatcattaattccaactttcgattcgtaggttactatcacgttcaatctttaaacggtaccgaaactcaacagtatgacttgaacgctattaacctagtatgccaagatgataataggccttcgtcctttgtccattggatagtaacagatccaaaagagttcaaatcattctcgatgtattacttggatgattcaatggttcaactcgtcaattccaatattcaacattacatttctaaaccattgccctatgaatttaaaaaccttctgtctgagaaaattgctatcgacacaatcctcaagcaatccaggctagaaaaagacttatccaccaaaaactcactgaagaaattaaacaatagttatatcgacattcattcctcactgaacgttctctataaatcagtcaataggcttattcgttacctcaaaaaatgctcaaaatcagaagtttcaattgactatgacacagttcaggaaatgaatactgtaatactgaaaattgaaaggcttaaattgataccccaagtcaaggaggagtttgacttagtgactctttcactactggtagacaatcttgatcagatggatcatcttttgtatctccggaaacaagtggaacagtacaaaatatctgaatcaatgtatagttagtcaatatcaaagtttcatacaacagtgaccatccagcatcgcatctgtcagtttcgtataatgtgcgcgcatgtgctgatcggtatatcaacatctcttcctcttctctgcttcctttttcctcttgtttgttatcaatgcagtacgtaggtagagccattggatcagtatccaaaacctggtcgtccattaacccggccacactgagtggtgctattgatatcatcgtggtggaacaagaaaatggtgatctggcttgctctcccttccatgttcggtttggaaagtttcaattattgaggccttcccaaaagaaagtggatttcatcgtcaatggagaaaagacagacttaccaatgaaactgggcgatgggggcgaagctttctttgtgtttgaaaccgatgctgccatcccgagtgagcttcaaacttcccccgtcatctcacctgtatccagcccagaaccagcctctccactgagtactccttccagaccaaactctgaaccagattatttagaattaggggatggagaatctacaaccagtgaattagagaacttcaagttaaatcgatatccctatttatcaactgaggtatcgcattcagatcctggtgttgggtctgtgagctcaagtcctgagaatacgaagattattcaaaagatctctcggaaactcaatacgaaaaatattccttccaaggttgataataacggaaatcttgtattggatatacaaggttacaaaagtgatgatctagacgacaattccaaatctttgaaacagttgttattggctgaactcggagaagatgtagatttggacaaggttattgaaaaagatcacgaaggaaacattatgatcaatggtgccatatctttgttgagcggagaagatgatctggaatcatttcctcagaccgacgaccaagctgagagtctcaaattggatctggagtcagataaatctgacattgaatcagacacaaatcatgaattgtctcgctacttcaaaacgctaagacttacatcagatcaattgaaatgtcttacactgaagaagggaattaacgagctgaaattcagtgtcaacaagggcaaatctgtggtgacggcaaatctatacttctgggactattatgaccccatagtcatatcggatatagatggaaccattactaaatctgatgccttaggccatgtatttaccatgattggccgtgattggactcacaaaggtgtagccaaattattttcagatatcaaatccaatggatacaatatcatgtatttgactgcaaggtctgttggtcaagccgattcgacccgctactatttaaacaacattgaacaagaaggcttgaggcttcctcaagggccggtgatcctttctccggatcgcacaatggccgctttacgaagagaggtaattttgaagaaacctgaggtgttcaagatggcatgtttaaacgacataaagaaactgtatctcaccaataccaaagacctaaatccgaacacagattctgcagatttcacggatatcaatacgaacactttgagatcttcaagtcttacagaggatgtacaaacgccattttacgccggttttggtaatagaattaccgatgctttgtcctataggtcagtagggataccatcatcaagaatttttaccataaacccagacggtgatgttcacatggagctactcgaattggctgggtatagaagttcttatgtacatattagtgagctggtagatcatttctttccaccagtgaacactgaactatttaagtcgatgccttctgatacctatcggaacacggcaaagttctctgatgtcaattattggaaagagcctttgtacaattttgaggagctcagtgatgaagattctagtgaagatgagctaagaagaagaaaggaggaggaacgtttacagtctgctcctcgtagtccaatattggcagcaggagcatcgtttttcaaaggaagtagtagccttttaggttcaccagaacggatgactttgagtgacccaaagccaactgaagttgcccctagcacaatcaaacccccaaaatcagttggttctgtatcatccgatgaagaaaagctgaaagatcatgatgatttcattgacgttgatcacgaagatgaaaccttggatgatgatgacgatccctttgactacgattacgagtatgaagatgaagaagaggaaaatgacgacgtagatgaagttgatgatggggaagagtattcagatgattattatgacgaagaagacgactacgatgaagaattagatcatactttagaaccagaccaaaagaaagaactggaccaaacagctgaagccaaccagttacccccttcaggacccgatgaaatggaatcaaaatctttcaagaaagccagtgatttaataagcaaaatgagaatcgatgacagctgagggtaaataagtattagttaaatctgcattttctatttatatacattatttctagagttctttggttacttcaactggaatggtggcgtccttttctctgtgtgactcggttttggcaaagatttttttaagaacagcgtcttcgtcgtcttcgtttctcataataaatatcttctttccatagctaaaccagtagaccagagaaacaaccaaccaaccaacaatagctgcagccgcgtagttcatgttggacttagtaactggcttcacaaatgggaatgagtagaataccagagcaaataatgcccatgcaacggtcatactggaggtgaaataaccaaactttccaatccagaatggaccatgagtgatactgtctcttccattgtatataagacaagagacaggaattgcataagacaacaggaggaaagtgatgcatccggttaccaaagagttataagccaagtctgaagccaagtaaaggcaactgaccaaggaacataaacagacggaaaacaaatgtgcgttgaatggaatacccattctcttgtcgacctttgaaaggaatggagaaaatggaagaccattgtctctggcaaaagaccaacagagtcgcatttgccaggtgtgaccagaaattaaacatccacaggcagtcaaaaacaacaagaaacccaggaaaatggcacctccttgattttgaagagcttggtagtaaatggaaataatgggggcaccggtagcagtgttcagcaatgcatccagatctctgatagagaagaacatcgaaatacagtacacaaaggaagtaaaaaatccaatggcaacagttcccatgatggcaattggaataaccctttcaggttgggcgacttcttcagccatatgggtagcacaatccagacacgagaaggaccaatttgggttaattaaaccaatgatgaaggcgattccagcggattcccaaccagtttggttgtcaaaatcggaaaac

SEQ ID NO: 4 - protein, 775 aa, Pichia pastoris
MQYVGRAIGSVSKTWSSINPATLSGAIDIIVVEQENGDLACSPFHVRFGKFQLLRPSQKKVDFIVNGEKTDLPMKLGDGGEAFFVFETDAAIPSELQTSPVISPVSSPEPASPLSTPSRPNSEPDYLELGDGESTTSELENFKLNRYPYLSTEVSHSDPGVGSVSSSPENTKIIQKISRKLNTKNIPSKVDNNGNLVLDIQGYKSDDLDDNSKSLKQLLLAELGEDVDLDKVIEKDHEGNIMINGAISLLSGEDDLESFPQTDDQAESLKLDLESDKSDIESDTNHELSRYFKTLRLTSDQLKCLTLKKGINELKFSVNKGKSVVTANLYFWDYYDPIVISDIDGTITKSDALGHVFTMIGRDWTHKGVAKLFSDIKSNGYNIMYLTARSVGQADSTRYYLNNIEQEGLRLPQGPVILSPDRTMAALRREVILKKPEVFKMACLNDIKKLYLTNTKDLNPNTDSADFTDINTNTLRSSSLTEDVQTPFYAGFGNRITDALSYRSVGIPSSRIFTINPDGDVHMELLELAGYRSSYVHISELVDHFFPPVNTELFKSMPSDTYRNTAKFSDVNYWKEPLYNFEELSDEDSSEDELRRRKEEERLQSAPRSPILAAGASFFKGSSSLLGSPERMTLSDPKPTEVAPSTIKPPKSVGSVSSDEEKLKDHDDFIDVDHEDETLDDDDDPFDYDYEYEDEEEENDDVDEVDDGEEYSDDYYDEEDDYDEELDHTLEPDQKKELDQTAEANQLPPSGPDEMESKSFKKASDLISKMRIDDS

SEQ ID NO: 5 - DNA, 858 bp, Yarrowia lipolytica
atgtctgctgcatctactggagctagcgccaccaagagtgcgcctgttagcagaaccgcccagggcactcagtctacacctgcttctctcaagagcgaaaccaagactaccgaaaccaattctaacatcaccgacaccaacggcgaagacctgttggcgttctggcataatggccggtttggcgccttcttcaagaaacacgaaatccccagaaagttcctccacacctccatcggcttcatcaccctatggctgtacacccaaggcatcaacctgaaccaggtcactcccgttctcgtggtgatggcattgttcatcggcacctttgacttcctgcgtctcagaaacaaggccctcaacaaggcctactgcaaagtcatgggagctctgatgcgagagtccgagattaacggcgtgaacggaaccatctggtacctgcttggcctcattctggtcttctcgttcgcctctaaggatgttgctcttatggctgttcttctgctctcctgggctgataccgcagcctcttccgtcggccgggcttatggatacctcactccgaagtacaagaacaagtcgctggctggttttatgggcgccttcattgcctctgagatcgcatgtctctttctatacggcatcatcatccccaagtacggcgctgaagtcgacacgcccggccagatcacctggtccaagcagtcttctcgacttactctgggagtcttgacagccatgtctggtctcgtcggagccgtttccgagtccattgatgtgttcggaatcgacgacaacttcaccattcccgtcctctcggcgctcttccttactggtctgctggacatcttcaagaagtaa

SEQ ID NO: 6 - protein, 285 aa, Yarrowia lipolytica
MSAASTGASATKSAPVSRTAQGTQSTPASLKSETKTTETNSNITDTNGEDLLAFWHNGRFGAFFKKHEIPRKFLHTSIGFITLWLYTQGINLNQVTPVLVVMALFIGTFDFLRLRNKALNKAYCKVMGALMRESEINGVNGTIWYLLGLILVFSFASKDVALMAVLLLSWADTAASSVGRAYGYLTPKYKNKSLAGFMGAFIASEIACLFLYGIIIPKYGAEVDTPGQITWSKQSSRLTLGVLTAMSGLVGAVSESIDVFGIDDNFTIPVLSALFLTGLLDIFKK

SEQ ID NO: 7 - DNA, 900 bp, Pichia pastoris
atggcagcaaccacagcacgtcctcagagaataggtcgctcgagagtgtcgaaattagaagaaagaatatcaacgtcttcctcctattcatctctctcgtcgcttgatgctagtgatgataccgtgttatccaccgagtcaacaagcccagtcttagataaggctaaaaatacatccacctcgtatttgttgttacaaagccacaagaggttcaataagttgattcataagcatgagttacccaggaaactattccatatgtccatcgggttcttcacattatacctgtacacaaagaatgtgcagaaagagcaaatccagttacccctggttgtgggatttgtagtggtgtttgcacttgatttaataaggttcagatggcctcaagttaacaaactttattgttccactgtggggttcctcatgagggagagagaggtggacgagtattataacggtgttaactggtacctgctcgggttggctgtagttttcttgtttttcaagaaggatattgcagtcatggcagtgctgttactaagtttatccgataccgctgcctccacggtcgggagagcttggggccagtatacacccaagatcaccagccacaaatctttagctggttcgttggccgcttgtgtcgttggaatattgtcatgctacctcttttatggatatttcgttccgaactatcccgaatgcaatgacattccaaaccagttcgaatgggtttccgaaaaaagtggcctcaacattcattcattggccctcctttctggattggtcgctagtgtaagtgaaggaattgatctcttcaactgggatgacaacttcaccattccagttctatcagctgtgtttttacgactggttatcaaactgacaaatacagactaa

SEQ ID NO: 8 - protein, 299 aa, Pichia pastoris
MAATTARPQRIGRSRVSKLEERISTSSSYSSLSSLDASDDTVLSTESTSPVLDKAKNTSTSYLLLQSHKRFNKLIHKHELPRKLFHMSIGFFTLYLYTKNVQKEQIQLPLVVGFVVVFALDLIRFRWPQVNKLYCSTVGFLMREREVDEYYNGVNWYLLGLAVVFLFFKKDIAVMAVLLLSLSDTAASTVGRAWGQYTPKITSHKSLAGSLAACVVGILSCYLFYGYFVPNYPECNDIPNQFEWVSEKSGLNIHSLALLSGLVASVSEGIDLFNWDDNFTIPVLSAVFLRLVIKLTNTD

SEQ ID NO: 9 - DNA, 600 bp, Saccharomyces cerevisiae
atggggaccgaagatgccattgcccttccaaatagcacgctagagccgcgtaccgaagctaagcaaagactatcatctaagagtcatcaagtctcggcgaaagtaacgattccagcaaaagaagaaattagtagtagcgatgacgatgcacacgttccagtgacagaaatacatttgaaatctcatgaatggttcggcgattttataactaaacatgaaattccccgtaaggtgttccattcttccattggctttattactttgtacctgtatacgcagggtattaattataaaaatgttttatggcctttgatatacgccttcatcatattgtttattttggatctgataagactaaactggccctttttcaatatgctttactgtagaactgtgggtgcgctaatgagaaaaaaggagattcatacatacaatggggtattgtggtacattcttgggttaatcttttcttttaactttttctctaaagatgttaccttaatatcgttatttttgctaagttggtccgatacagccgccgcaactattggaagaaagtatggtcatttaacacccaaagtggcaagaaataaatccctt

SEQ ID NO: 10 - protein, 290 aa, Saccharomyces cerevisiae
MGTEDAIALPNSTLEPRTEAKQRLSSKSHQVSAKVTIPAKEEISSSDDDAHVPVTEIHLKSHEWFGDFITKHEIPRKVFHSSIGFITLYLYTQGINYKNVLWPLIYAFIILFILDLIRLNWPFFNMLYCRTVGALMRKKEIHTYNGVLWYILGLIFSFNFFSKDVTLISLFLLSWSDTAAATIGRKYGHLTPKVARNKSLAGSIAAFTVGVITCWVFYGYFVPAYSYVNKPGEIQWSPETSRLSLNMLSLLGGVVAALSEGIDLFNWDDNFTIPVLSSLFMNAVIKTFKK

SEQ ID NO: 11 - protein, 5 aa, Artificial Sequence
  catalytic sequence
DIDGT

SEQ ID NO: 12 - DNA, 30 bp, Artificial Sequence
  Primer
gcggccgcgaagacggtgagtatggccatc

SEQ ID NO: 13 - DNA, 30 bp, Artificial Sequence
  Primer
gcggccgcccaaaccatgcatacaaatcag

SEQ ID NO: 14 - DNA, 44 bp, Artificial Sequence
  Primer
aaaaacaactaattattcgaaatggcagcaaccacagcacgtcc

SEQ ID NO: 15 - DNA, 45 bp, Artificial Sequence
  Primer
aaggcgaattaattcgcggccgcttagtctgtatttgtcagtttg

SEQ ID NO: 16 - DNA, 26 bp, Artificial Sequence
  Primer
ttcgaaatgggtgcgagagcgtcagt

SEQ ID NO: 17 - DNA, 26 bp, Artificial Sequence
  Primer
gcggccgcttattgtgacgaggggtc

SEQ ID NO: 18 - DNA, 40 bp, Artificial Sequence
  Primer
tagggataacagggtaatccattgacacagaactcgacct

SEQ ID NO: 19 - DNA, 40 bp, Artificial Sequence
  Primer
attaccctgttatccctaccgtactgtacaccgtagtttg

SEQ ID NO: 20 - DNA, 34 bp, Artificial Sequence
  Primer
agatcttaataagccacactgagtggtgctattg

SEQ ID NO: 21 - DNA, 35 bp, Artificial Sequence
  Primer
gcggccgcttacgtattctcaggacttgagctcac